(19) United States
(12) Patent Application Publication
(10) Pub. No.: US 2021/0046071 A1
(43) Pub. Date: Feb. 18, 2021

(54) TETRACYCLIC COMPOUNDS AND THEIR SALTS, COMPOSITIONS, AND METHODS FOR THEIR USE

(71) Applicant: Senhwa Biosciences, Inc., New Taipei City (TW)

(72) Inventor: John SOONG, Chino, CA (US)

(21) Appl. No.: 16/993,840

(22) Filed: Aug. 14, 2020

Related U.S. Application Data
(60) Provisional application No. 62/946,774, filed on Dec. 11, 2019.
(60) Provisional application No. 62/886,621, filed on Aug. 14, 2019.

(51) Int. Cl. A61K 31/497; A61K 9/00; A61K 47/26; A61P 35/04

(52) U.S. Cl. CPC A61K 31/497; A61P 35/04; A61K 47/26; A61K 9/0019

(57) ABSTRACT

The present invention includes 2-(4-Methyl-[1,4]diazepan-1-yl)-5-oxo-5H-7-thia-1,11b-diaza-benzo[c]fluorene-6-carboxylic acid (5-methyl-pyrazin-2-ylmethyl)-amide (Compound I) or a pharmaceutically acceptable salt thereof for use in treating cancer with PALB2 mutation and/or BRCA2 mutation.

### CROSS-REFERENCE TO RELATED APPLICATIONS

This application claims the priority benefit of U.S. Provisional Application No. 62/886,621, filed Aug. 14, 2019 and U.S. Provisional Application No. 62/946,774, filed Dec. 11, 2019, the disclosures of which are incorporated by reference herein in their entireties.

### FIELD OF THE DISCLOSURE

The present invention generally relates to fused tetracyclic compound or a pharmaceutically acceptable salts thereof, pharmaceutical composition containing them, and methods of their use in treating diseases or disorders including cancer.

### BACKGROUND OF THE DISCLOSURE

A variety of tetracyclic quinolone compounds or napththyridinone fused tetracyclic compounds have been suggested to function by interacting with quadruplex-forming regions of nucleic acids and modulating ribosomal RNA transcription. See, for example, U.S. Pat. Nos. 7,928,100 and 8,853,234. Specifically, the tetracyclic quinolone compounds can stabilize the DNA G-quadruplexes (G4s) in cancer cells and thereby induce synthetic lethality in cancer cells. Since treatment of cells with G4-stabilizing agents can lead to the formation of DNA double strand breaks (DSBs), DSB formation induced by G4-stabilizing ligand/agent (such as the tetracyclic quinolones) treatment would be more pronounced in cells genetically deficient in, or chemically inhibited in, repair pathways including both non-homologous end joining (NHEJ) and homologous recombination (HR) repair. Furthermore, the tetracyclic quinolone compounds selectively inhibit rRNA synthesis by RNA polymerase I (Pol I) in the nucleolus, but do not inhibit mRNA synthesis by RNA polymerase II (Pol II) and do not inhibit DNA replication or protein synthesis. It is suggested that targeting RNA polymerase I (Pol I) to activate p53 through the nucleolar stress pathway may results in selective activation of p53 in tumor cells. The p53 protein normally functions as a tumor suppressor by causing cancer cells to self-destruct. Activating p53 to kill cancer cells is a well validated anticancer strategy and many approaches are being employed to exploit this pathway. Selective activation of p53 in tumor cells would be an attractive method of treating, controlling, ameliorating tumor cells while not affecting normal healthy cells. The aforementioned tetracyclic quinolones are disclosed in U.S. Pat. Nos. 7,928,100 and 8,853,234, and the contents of this publication are herein incorporated by reference in their entirety for all intended purposes.

### SUMMARY OF THE DISCLOSURE

In one embodiment of the present invention provides methods for treating cancer in a subject in need thereof, comprising administering a therapeutically effective amount of Compound I.

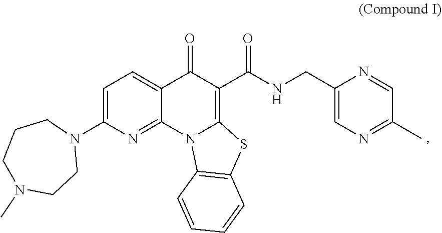

or a pharmaceutically acceptable salt and/or solvate thereof, wherein the subject has PALB2 mutation and/or BRCA2 mutation.

In one embodiment of any one of the methods disclosed herein, the subject has a PALB2 mutation. In some embodiments, the subject has a BRCA2 mutation. In some embodiments, the subject has a PALB2 mutation and a BRCA2 mutation. In other embodiments, the subject has one or more additional gene mutations in the homologous recombination pathway.

In one embodiment of any one of the methods for treating cancer disclosed herein, the cancer is a solid tumor. In one embodiment, the cancer is a hematologic malignancy, colorectal cancer, breast cancer, lung cancer, liver cancer, ovarian cancer, cervical cancer, Ewing's sarcoma, pancreatic cancer, cancer of the lymph nodes, colon cancer, prostate cancer, brain cancer, bone cancer, cancer of the head and neck, skin cancer, kidney cancer, osteosarcoma, cancer of the heart, uterine cancer, gastrointestinal malignancies, or carcinomas of the larynx and oral cavity. In one embodiment, the cancer is breast cancer, ovarian cancer, or pancreatic cancer. In some embodiments, the hematologic malignancy is selected from leukemia, lymphoma, myeloma, and multiple myeloma.

In one embodiment of any one of the methods for treating cancer disclosed herein, the cancer is a PALB2-mutated cancer. In one embodiment, the cancer is a BRCA2-mutated cancer. In one embodiment, the PALB2-mutated cancer or the BRACA2-mutated cancer is breast cancer, ovarian cancer, pancreatic cancer, or prostate cancer. In one embodiment, the PALB2-mutated cancer or the BRACA2-mutated cancer is breast cancer or prostate cancer. In some embodiments, the BRCA2-mutated cancer or the PALB2-mutated cancer is breast cancer or ovarian cancer. In some embodiments, the BRCA2-mutated cancer or the PALB2-mutated cancer is breast cancer.

In one embodiment of any one of the methods disclosed herein, the PALB2 mutation is a loss-of-function mutation of the PALB2 gene. In one embodiment, the PALB2 mutation is a monoallelic loss-of-function mutation. In other embodiments, the PALB2 mutation is a biallelic loss-of-function mutation.

In one embodiment of any one of the methods disclosed herein, the BRCA2 mutation is a loss-of-function mutation of the BRCA2 gene.

In one embodiment of any one of the methods disclosed herein, the subject is administered the Compound I or a pharmaceutically acceptable salt thereof at a dose ranging from about 50 mg to about 1000 mg of the Compound I or a pharmaceutically acceptable salt and/or solvate thereof, per body surface area of the subject (m²). In other embodiments, the subject is administered the Compound I or a pharmaceutically acceptable salt thereof at a dose ranging from about 50 mg to about 650 mg of the Compound I or a pharmaceutically acceptable salt and/or solvate thereof, per body surface area of the subject (m²). In one embodiment, the dose ranges from about 150 mg to about 650 mg of the Compound I or a pharmaceutically acceptable salt and/or solvate thereof, per body surface area of the subject (m²). In some embodiments, the subject is administered the Compound I or a pharmaceutically acceptable salt and/or solvate thereof at a dose of about 50 mg, about 100 mg, about 150 mg, about 170 mg, about 200 mg, about 250 mg, about 325 mg, about 475 mg, or about 650 mg of the Compound I or a pharmaceutically acceptable salt and/or solvate thereof, per body surface area of the subject (m²).

In one embodiment of any one of the methods as disclosed herein, the subject is administered the Compound I or a pharmaceutically acceptable salt and/or solvate thereof at a dose of at least 100 mg of the Compound I or a pharmaceutically acceptable salt and/or solvate thereof, per body surface area of the subject (m²). In one embodiment of any one of the methods for treating cancer disclosed herein, the subject is administered the Compound I or a pharmaceutically acceptable salt and/or solvate thereof at a dose of at least 150 mg of the Compound I or a pharmaceutically acceptable salt and/or solvate thereof, per body surface area of the subject (m²). In one embodiment, the subject is administered the Compound I or a pharmaceutically acceptable salt and/or solvate thereof at a dose ranging from about 150 mg to about 800 mg of the Compound I or a pharmaceutically acceptable salt and/or solvate thereof, per body surface area of the subject (m²). In one embodiment, the subject has a BRCA2 mutation.

In one embodiment of any one of the methods for treating cancer disclosed herein, the subject is administered the Compound I or a pharmaceutically acceptable salt and/or solvate thereof at a dose of at least 650 mg of the Compound I or a pharmaceutically acceptable salt and/or solvate thereof, per body surface area of the subject (m²). In one embodiment, the subject is administered the Compound I or a pharmaceutically acceptable salt and/or solvate thereof at a dose ranging from about 650 mg to about 800 mg of the Compound I or a pharmaceutically acceptable salt and/or solvate thereof, per body surface area of the subject (m²). In one embodiment, the subject has a PALB2 mutation.

In one embodiment of any one of the methods for treating cancer disclosed herein, the Compound I or a pharmaceutically acceptable salt thereof is administered as a solid dosage form or a liquid dosage form. In some embodiments, the Compound I or a pharmaceutically acceptable salt thereof is in a lyophilized form. In other embodiments, the Compound I or a pharmaceutically acceptable salt thereof is administered in a liquid dosage form prepared from a lyophilized form of the Compound I or a pharmaceutically acceptable salt thereof. In one embodiment, the Compound I or a pharmaceutically acceptable salt thereof is administered intravenously.

In one embodiment of any one of the methods for treating cancer disclosed herein, the Compound I or a pharmaceutically acceptable salt thereof is administered in a 28-day cycle. In one embodiment, a dose of the Compound I or a pharmaceutically acceptable salt thereof is administered on day 1, day 8, and day 15 of the 28-day cycle. In other embodiments, a dose of the Compound I or a pharmaceutically acceptable salt thereof is administered on day 1 and day 8 of the 28-day cycle.

In one embodiment, the present disclosure provides pharmaceutical compositions comprising Compound I, or a pharmaceutically acceptable salt and/or solvate thereof and a pharmaceutically acceptable carrier or excipient, wherein, the composition provides a plasma Compound I AUC_{∞} ranging from about 2,000 ng*hr/mL to about 110,000 ng*hr/mL after a single dose administration of the composition to a human subject at a dose of about 50 mg to about 650 mg of the Compound I or a pharmaceutically acceptable salt and/or solvate thereof, per body surface area of the subject (m²).

In one embodiment of any one of the pharmaceutical compositions as disclosed herein, the dose of Compound I, or a pharmaceutically acceptable salt or solvate thereof ranges from about 150 mg² to about 650 mg of the Compound I or a pharmaceutically acceptable salt and/or solvate thereof, per body surface area of the subject (m²).

In one embodiment of any one of the pharmaceutical compositions as disclosed herein, the plasma Compound I AUC_{∞} ranges from about 5,000 ng*hr/mL to about 70,000 ng*hr/mL.

In one embodiment of any one of the pharmaceutical compositions as disclosed herein, the composition provides a plasma Compound I Cₘₐₓ ranging from about 200 ng/mL to about 6,000 ng/mL. In some embodiments, the composition provides a plasma Compound I Cₘₐₓ ranging from about 500 ng/mL to about 5,000 ng/mL.

In one embodiment of any one of the pharmaceutical compositions as disclosed herein, the composition provides a plasma Compound I Tₘₐₓ ranging from about 0.3 hour to about 2.0 hours. In some embodiments, the composition provides a plasma Compound I Tₘₐₓ ranging from about 0.4 hour to about 1.5 hour.

In one embodiment of any one of the pharmaceutical compositions as disclosed herein, the composition provides a plasma Compound I Cₘₐₓ ranging from about 200 ng/mL to about 6,000 ng/mL and wherein the composition provides a plasma Compound I Tₘₐₓ ranging from about 0.3 hour to about 2.0 hours. In some embodiments, the composition provides a plasma Compound I Cₘₐₓ ranging from about 500 ng/mL to about 5,000 ng/mL and wherein the composition provides a plasma Compound I Tₘₐₓ ranging from about 0.4 hour to about 1.5 hour.

In one embodiment of any one of the pharmaceutical compositions as disclosed herein, the plasma Compound I AUC_{∞} ranges from about 11,000 ng*hr/mL to about 52,000 ng*hr/mL after a single dose administration of the composition to a human subject at a dose of about 475 mg of the Compound I or a pharmaceutically acceptable salt and/or solvate thereof, per body surface area of the subject (m²). In one embodiment, the plasma Compound I AUC_{∞} ranges from about 30,000 ng*hr/mL to about 40,000 ng*hr/mL after a single dose administration of the composition to a human subject at a dose of about 475 mg of the Compound I or a pharmaceutically acceptable salt and/or solvate thereof, per body surface area of the subject (m²).

In one embodiment of any one of the pharmaceutical compositions as disclosed herein, the plasma Compound I Cₘₐₓ ranges from about 900 ng/mL to about 2,600 ng/mL after a single dose administration of the composition to a human subject at a dose of about 475 mg of the Compound I or a pharmaceutically acceptable salt and/or solvate thereof, per body surface area of the subject (m²). In one embodiment, the plasma Compound I Cₘₐₓ ranges from about 1,200 ng/mL to about 2,300 ng/mL after a single dose administration of the composition to a human subject at a dose of about 475 mg of the Compound I or a pharmaceutically acceptable salt and/or solvate thereof, per body surface area of the subject (m²).

In one embodiment of any one of the pharmaceutical compositions as disclosed herein, the plasma Compound I Tₘₐₓ ranges from about 1.0 hour to about 1.4 hour after a single dose administration of the composition to a human subject at a dose of about 475 mg of the Compound I or a pharmaceutically acceptable salt and/or solvate thereof, per body surface area of the subject (m²).

In one embodiment of any one of the pharmaceutical compositions as disclosed herein, the composition is a solid composition or a liquid composition. In one embodiment, the composition comprises a lyophilized form of Compound I or a pharmaceutically acceptable salt and/or solvate thereof. In one embodiment, the composition comprises is a liquid composition prepare with a lyophilized form of Compound I or a pharmaceutically acceptable salt and/or solvate thereof. In one embodiment, the composition is for an intravenous administration.

In one embodiment of any one of the pharmaceutical compositions as disclosed herein, the composition comprises less than about 1% impurities. In some embodiments, the composition comprises less than 0.1% of

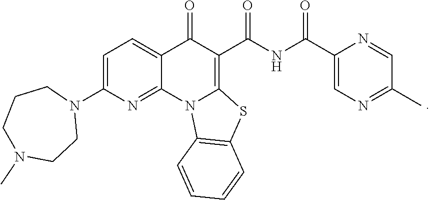

In one embodiment of any one of the pharmaceutical compositions as disclosed herein, the pharmaceutically acceptable excipient is a bulking agent. In one embodiment, the bulking agent is sucrose, mannitol, or trehalose.

In one embodiment of the present disclosure, methods for treating or ameliorating cell proliferation disorder in a subject is disclosure, wherein the method comprising administering to a subject in need thereof a therapeutically effective amount of any one of the pharmaceutical composition as disclosed herein. In one embodiment, the cell proliferation disorder is a cancer.

In one embodiment of any one of the methods as disclosed herein, cancer is a solid tumor. In one embodiment, the cancer is a hematologic malignancy, colorectal cancer, breast cancer, lung cancer, liver cancer, ovarian cancer, cervical cancer, Ewing's sarcoma, pancreatic cancer, cancer of the lymph nodes, colon cancer, prostate cancer, brain cancer, bone cancer, cancer of the head and neck, skin cancer, kidney cancer, osteosarcoma, cancer of the heart, uterine cancer, gastrointestinal malignancies, or carcinomas of the larynx and oral cavity. In some embodiments, the cancer is breast cancer, ovarian cancer, or pancreatic cancer. In other embodiments, the hematologic malignancy is selected from leukemia, lymphoma, myeloma, and multiple myeloma.

In one embodiment of any one of the methods as disclosed herein, the cancer is a PALB2-mutated cancer. In other embodiments, the cancer is a BRCA2-mutated cancer. In one embodiment, the PALB2-mutated cancer or the BRCA2-mutated cancer is breast cancer, ovarian cancer, pancreatic cancer, or prostate cancer. In one embodiment, the PALB2-mutated cancer or the BRCA2-mutated cancer is breast cancer or prostate cancer. In some embodiments, the BRCA2-mutated cancer or the PALB2-mutated cancer is breast cancer or ovarian cancer. In some embodiments, the BRCA2-mutated cancer or the PALB2-mutated cancer is breast cancer.

In one embodiment of any one of the methods as disclosed herein, the PALB2 mutation is a loss-of-function mutation of the PALB2 gene. In one embodiment, the PALB2 mutation is a monoallelic loss-of-function mutation. In other embodiments, the PALB2 mutation is a biallelic loss-of-function mutation.

In one embodiment of any one of the methods as disclosed herein, the BRCA2 mutation is a loss-of-function mutation of the BRCA2 gene.

In one embodiment of any one of the methods as disclosed herein, the subject is administered the Compound I or a pharmaceutically acceptable salt thereof at a dose ranging from about 50 mg to about 1,000 mg of the Compound I or a pharmaceutically acceptable salt and/or solvate thereof, per body surface area of the subject (m²). In one embodiment, the subject is administered the Compound I or a pharmaceutically acceptable salt thereof at a dose ranging from about 50 mg to about 650 mg of the Compound I or a pharmaceutically acceptable salt and/or solvate thereof, per body surface area of the subject (m²). In some embodiments, the dose ranges from about 150 mg to about 650 mg of the Compound I or a pharmaceutically acceptable salt and/or solvate thereof, per body surface area of the subject (m²). In one embodiment, the subject is administered the Compound I or a pharmaceutically acceptable salt and/or solvate thereof at a dose of about 50 mg, about 100 mg, about 150 mg, about 170 mg, about 200 mg, about 250 mg, about 325 mg, about 475 mg, or about 650 mg of the Compound I or a pharmaceutically acceptable salt and/or solvate thereof, per body surface area of the subject (m²).

In one embodiment of any one of the methods as disclosed herein, the subject is administered the Compound I or a pharmaceutically acceptable salt and/or solvate thereof at a dose of at least 100 mg of the Compound I or a pharmaceutically acceptable salt and/or solvate thereof, per body surface area of the subject (m²). In one embodiment of any one of the methods as disclosed herein, the subject is administered the Compound I or a pharmaceutically acceptable salt and/or solvate thereof at a dose of at least 150 mg of the Compound I or a pharmaceutically acceptable salt and/or solvate thereof, per body surface area of the subject (m²). In one embodiment, the subject is administered the Compound I or a pharmaceutically acceptable salt and/or solvate thereof at a dose ranging from about 150 mg to about 800 mg of the Compound I or a pharmaceutically acceptable salt and/or solvate thereof, per body surface area of the subject (m²). In one embodiment, the subject has a BRCA2 mutation.

In one embodiment of any one of the methods as disclosed herein, the subject is administered the Compound I or a pharmaceutically acceptable salt and/or solvate thereof at a dose of at least 650 mg of the Compound I or a pharmaceutically acceptable salt and/or solvate thereof, per body surface area of the subject (m²). In some embodiments, the subject is administered the Compound I or a pharmaceutically acceptable salt and/or solvate thereof at a dose ranging from about 650 mg to about 800 mg of the Compound I or a pharmaceutically acceptable salt and/or solvate thereof, per body surface area of the subject (m²). In one embodiment, the subject has a PALB2 mutation.

In one embodiment of any one of the methods as disclosed herein, the Compound I or a pharmaceutically acceptable salt thereof is administered intravenously.

In one embodiment of any one of the methods as disclosed herein, the composition is administered in a 28-day cycle. In one embodiment, a dose of the Compound I or a pharmaceutically acceptable salt thereof is administered on day 1, day 8, and day 15 of the 28-day cycle. In other embodiments, a dose of the Compound I or a pharmaceutically acceptable salt thereof is administered on day 1 and day 8 of the 28-day cycle.

In one embodiment, the present disclosure relates to methods of inhibiting Pol I transcription in a subject, comprising administering to a subject in need thereof a therapeutically effective amount of any one of pharmaceutical compositions comprising Compound I or a pharmaceutically acceptable salt thereof, as disclosed herein. In one embodiment, the inhibiting Pol I transcription is in peripheral blood mononuclear cells.

In one embodiment, the present disclosure relates to methods of stabilizing G-quadruplexes (G4s) in a subject, comprising administering to a subject in need thereof a therapeutically effective amount of any one of pharmaceutical compositions comprising Compound I or a pharmaceutically acceptable salt thereof, as disclosed herein. In one embodiment, the stabilizing G4s is in peripheral blood mononuclear cells.

### BRIEF DESCRIPTION OF THE DRAWINGS

FIG. 1A is a plot of individual Cₘₐₓ versus actual dose administered on Day 1 for all subjects in Groups 1-7. A zoomed-in view is presented due to a possible PK outlier. FIG. 1B is a plot of individual Cₘₐₓ versus actual dose administered on Day 8 for all subjects in Groups 1-7.

FIG. 1C is a plot of individual AUC_{∞} versus actual dose administered on Day 1 for all subjects in Groups 1-7. FIG. 1D is a plot of individual AUC_{∞} versus actual dose administered on Day 8 for all subjects in Groups 1-7.

FIG. 2A is a plot of individual Cₘₐₓ versus actual dose administered on Day 1 for all subjects in Groups 8-10. FIG. 2B is a plot of individual Cₘₐₓ versus actual dose administered on Day 15 for all subjects in Groups 8-10.

FIG. 2C is a plot of individual AUC_{∞} versus actual dose administered on Day 1 for all subjects in Groups 8-10. FIG. 2D is a plot of individual AUC_{∞} versus actual dose administered on Day 15 for all subjects in Groups 8-10.

FIG. 3A is a plot of individual dose-normalized Cₘₐₓ versus actual dose administered on Day 8 for all subjects in Groups 1-7. FIG. 3B is a plot of individual dose-normalized Cₘₐₓ versus actual dose administered on Day 15 for all subjects in Groups 8-10.

FIG. 3C is a plot of individual dose-normalized AUC_{∞} versus actual dose administered on Day 8 for all subjects in Groups 1-7. FIG. 3D is a plot of individual dose-normalized AUC_{∞} versus actual dose administered on Day 15 for all subjects in Groups 8-10.

FIG. 4 shows best % tumor shrinkage from baseline in all patients with BRCA1 or BRCA2 mutation from study described in Example 3.

FIG. 5 shows best % tumor shrinkage from baseline in all breast cancer patients with BRCA1 or BRCA2 mutation in patients from study described in Example 3.

FIG. 6 shows best % tumor shrinkage from baseline in all breast cancer patients with BRCA2 mutation from study described in Example 3.

FIG. 7 shows best % tumor shrinkage from baseline for evaluable patients with genetic mutations labelled from the study described in Example 3.

FIG. 8 shows duration on therapy for all patients at each dose level with genetic mutations labelled from the study described in Example 3.

FIG. 9 depicts CT scans of a patient from Group 10 (650 mg/m²) who harbored a PALB2 mutation and showed partial response (PR), A) prior to treatment with Compound I and B) 6-month follow-up scan following treatment with Compound I.

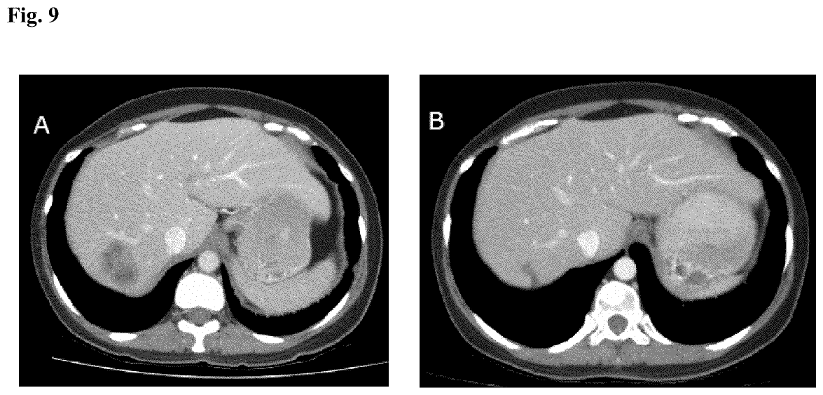

### DETAILED DESCRIPTIONS OF THE DISCLOSURE

The present invention relates to 2-(4-Methyl-[1,4]diazepan-1-yl)-5-oxo-5H-7-thia-1,11b-diaza-benzo[c]fluorene-6-carboxylic acid (5-methyl-pyrazin-2-ylmethyl)-amide (Compound I) or a pharmaceutically acceptable salts or solvates thereof. Compound I or a pharmaceutically acceptable salts or solvates thereof can stabilize G-quadruplexes (G4s) and/or inhibit Pol I and can be useful for treating disorders characterized by proliferation of cells.

### Definitions

It is to be understood that the terminology used herein is for the purpose of describing particular embodiments only and is not intended to be limiting.

Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

Following long-standing patent law convention, the terms “a”, “an”, and “the” refer to “one or more” when used in this application, including the claims. Thus, for example, reference to “a carrier” includes mixtures of one or more carriers, two or more carriers, and the like.

The term “compound(s) of the present invention” or “present compound(s)” refers to 2-(4-Methyl-[1,4]diazepan-1-yl)-5-oxo-5H-7-thia-1,11b-diaza-benzo[c]fluorene-6-carboxylic acid (5-methyl-pyrazin-2-ylmethyl)-amide (Compound I) or isomers, salts, N-oxides, sulfoxides, sulfones, or solvates thereof.

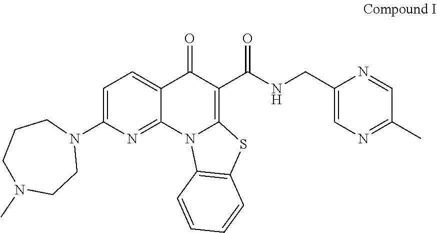

The term “isomer” refers to compounds having the same chemical formula but may have different stereochemical formula, structural formula, or special arrangements of atoms. Examples of isomers include stereoisomers, diastereomers, enantiomers, conformational isomers, rotamers, geometric isomers, and atropisomers.

“N-oxide”, also known as amine oxide or amine-N-oxide, means a compound that derives from a compound of the present invention via oxidation of an amine group of the compound of the present invention. An N-oxide typically contains the functional group R₃N⁺—O⁻ (sometimes written as R₃N═O or R₃N→O).

The term “ester” refers to any ester of a compound of the present invention in which any of the —COOH functions of the molecule is replaced by a —COOR function, in which the R moiety of the ester is any carbon-containing group which forms a stable ester moiety, including but not limited to alkyl, alkenyl, alkynyl, cycloalkyl, cycloalkylalkyl, aryl, arylalkyl, heterocyclyl, heterocyclylalkyl and substituted derivatives thereof. The term “ester” includes but is not limited to pharmaceutically acceptable esters thereof. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids.

“Sulfoxide” refers to a compound that derived from a compound of the present invention via oxidation of a sulfur (S) atom. Sulfoxides are commonly written as —S(═O)—, —S(O)—, or —(S→O)—. “Sulfone” refers to a compound that derived from a compound of the present invention via further oxidation of a sulfur atom. Sulfones are commonly written as —S(═O)₂—, —S(O)₂—, or —(S(→O)₂)—.

The term “room temperature” as used herein, means from 21 degrees Celsius to 27 degrees Celsius.

The term “composition” denotes one or more substance in a physical form, such as solid, liquid, gas, or a mixture thereof. One example of composition is a pharmaceutical composition, i.e., a composition related to, prepared for, or used in medical treatment. The term “formulation” is also used to indicate one or more substance in a physical form, such as solid, liquid, gas, or a mixture thereof.

The term “co-administration” or “coadministration” refers to administration of a formulation or a composition comprising Compound I, or a pharmaceutically acceptable salt or solvate thereof; and (b) one or more additional therapeutic agent and/or radio therapy, in combination, i.e., together in a coordinated fashion.

The term “carboxylic acid” refers to an organic acid characterized by one or more carboxyl groups, such as acetic acid and oxalic acid. “Sulfonic acid” refers to an organic acid with the general formula of R—(S(O)₂—OH)ₙ, wherein R is an organic moiety and n is an integer above zero, such as 1, 2, and 3. The term “polyhydroxy acid” refers to a carboxylic acid containing two or more hydroxyl groups. Examples of polyhydroxy acid include, but are not limited to, lactobionic acid, gluconic acid, and galactose.

As used herein, “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

“Salts” include derivatives of an active agent, wherein the active agent is modified by making acid or base addition salts thereof. Preferably, the salts are pharmaceutically acceptable salts. Such salts include, but are not limited to, pharmaceutically acceptable acid addition salts, pharmaceutically acceptable base addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine dicyclohexylamine and the like. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Standard methods for the preparation of pharmaceutically acceptable salts and their formulations are well known in the art, and are disclosed in various references, including for example, “Remington: The Science and Practice of Pharmacy”, A. Gennaro, ed., 20th edition, Lippincott, Williams & Wilkins, Philadelphia, Pa.

As used herein, “solvate” means a complex formed by solvation (the combination of solvent molecules with molecules or ions of the compounds of the present invention), or an aggregate that consists of a solute ion or molecule (the compounds of the present invention) with one or more solvent molecules. In the present invention, the preferred solvate is hydrate. Examples of hydrate include, but are not limited to, hemihydrate, monohydrate, dihydrate, trihydrate, hexahydrate, etc. It should be understood by one of ordinary skill in the art that the pharmaceutically acceptable salt of the present compound may also exist in a solvate form. The solvate is typically formed via hydration which is either part of the preparation of the present compound or through natural absorption of moisture by the anhydrous compound of the present invention. Solvates including hydrates may be consisting in stoichiometric ratios, for example, with two, three, four salt molecules per solvate or per hydrate molecule. Another possibility, for example, that two salt molecules are stoichiometric related to three, five, seven solvent or hydrate molecules. Solvents used for crystallization, such as alcohols, especially methanol and ethanol; aldehydes; ketones, especially acetone; esters, e.g. ethyl acetate; may be embedded in the crystal grating. Preferred are pharmaceutically acceptable solvents.

The term “substantially similar” as used herein with regards to bioavailability of pharmacokinetics means that the two or more therapeutically active agents or drugs provide the same therapeutic effects in a subject.

The term “substantially free of” as used herein, means free from therapeutically effective amounts of compounds when administered in suggested doses, but may include trace amounts of compounds in non-therapeutically effective amounts.

The terms “excipient”, “carrier”, and “vehicle” are used interchangeably throughout this application and denote a substance with which a compound of the present invention is administered.

“Therapeutically effective amount” means the amount of a therapeutically active agent, when administered to a patient for treating a disease or other undesirable medical condition, is sufficient to have a beneficial effect with respect to that disease or condition. The therapeutically effective amount will vary depending on the therapeutically active agent, the disease or condition and its severity, and the age, weight, etc. of the patient to be treated. Determining the therapeutically effective amount of the therapeutically active agent is within the ordinary skill of the art and requires no more than routine experimentation.

As used herein, the terms “additional pharmaceutical agent” or “additional therapeutic agent” or “additional therapeutically active agent” with respect to the compounds described herein refers to an active agent other than the Compound I or a pharmaceutically acceptable salt or solvate thereof, which is administered to elicit a therapeutic effect. The pharmaceutical agent(s) may be directed to a therapeutic effect related to the condition that the compounds of the present disclosure is intended to treat or ameliorate (e.g., cancer) or, the pharmaceutical agent may be intended to treat or ameliorate a symptom of the underlying condition (e.g., tumor growth, hemorrhage, ulceration, pain, enlarged lymph nodes, cough, jaundice, swelling, weight loss, cachexia, sweating, anemia, paraneoplastic phenomena, thrombosis, etc.) or to further reduce the appearance or severity of side effects of the compounds of the present disclosure.

As used herein, the phrase “a disorder characterized by cell proliferation” or “a condition characterized by cell proliferation” include, but are not limited to, cancer, benign and malignant tumors. Examples of cancer and tumors include, but are not limited to, cancers or tumor growth of the large intestine, breast (including inflammatory breast cancer), lung, liver, pancreas, lymph node, colon, rectum, prostate, brain, head and neck, skin, kidney, osteosarcoma, blood and heart (e.g., leukemia, lymphoma, and carcinoma).

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

The term “patient” or “subject” as used herein, includes humans and animals, preferably mammals.

As used herein, the terms “inhibiting” or “reducing” cell proliferation is meant to slow down, to decrease, or, for example, to stop the amount of cell proliferation, as measured using methods known to those of ordinary skill in the art, by, for example, 10%, 20%, 30%, 40%, 50%, 60%, 70%, 80%, 90%, 95%, or 100%, when compared to proliferating cells that are not subjected to the methods and compositions of the present application.

As used herein, the term “apoptosis” refers to an intrinsic cell self-destruction or suicide program. In response to a triggering stimulus, cells undergo a cascade of events including cell shrinkage, blebbing of cell membranes and chromatic condensation and fragmentation. These events culminate in cell conversion to clusters of membrane-bound particles (apoptotic bodies), which are thereafter engulfed by macrophages.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the present application.

Compound I

Compound I is a synthetically derived small molecule, which can selectively binds and stabilizes DNA G-quadruplex (G4) structures. Key attributes of Compound I include induction of DNA damage through G4 stabilization which is dependent on intact BRCA12 and other homologous recombination mediated pathways for resolution. Cumulative mutations affecting BRCA12 and homologous recombination (HR) deficient tumor cells result in synthetic lethality.

Compound I showed specific toxicity against BRCA12 deficient cells in a number of cell lines of different genetic backgrounds (colon, breast and ovary) and different specifies origin (mouse and human). Compound I exhibited a wide therapeutic index of activity in BRCA2 knockout tumor cells in a xenograft model, when compared with isogenic wild type control cells. Without bound to any theory, the data to date attribute the anti-tumor activity of Compound I to bind and stabilize G4 DNA structure and impede the progression of DNA replication complexes and induces single stranded DNA gaps or breaks. The BRCA pathway is required for the repair of Compound I induced DNA damage, and that compromised DNA damage repair in the absence of BRCA genes will lead to lethality. BRCA deficient cells can be killed by Compound I at low drug concentration which are not effective at inhibiting rDNA transcription which suggests, without bound to any theory, that the dose used to treat BRCA deficient cancers is lower than that required to inhibit RNA Polymerase I and disrupt nucleons function.

Further, Compound I has shown to be responsive to PALB2 mutated cancers. The PALB2 gene is called the partner and localizer of the BRCA2 gene. It provides instructions to make a protein that works with the BRCA2 protein to repair damaged DNA and stop tumor growth. Inheriting two abnormal PALB2 genes causes Fanconi anemia type N, which suppresses bone marrow function and leads to extremely low levels of red blood cells, white blood cells, and platelets.

In some embodiments, Compound I is free base. In other embodiments, Compound I is provided as a pharmaceutically acceptable salt. In one embodiment, the salt is a hydrochloric acid addition salt, a sulfuric acid addition salt, a sulfonic acid addition salt, a carboxylic acid addition salt, or a polyhydroxy acid addition salt.

Compound I exhibited antiproliferation activity against a variety of cancer cell lines. See Example 2.

Pharmaceutical Formulations

In one embodiment, the present invention provides a pharmaceutical composition comprising a therapeutically effective amount of a Compound I, or a pharmaceutically acceptable salt, ester, and/or solvate thereof, as disclosed herein, as the active ingredient, combined with a pharmaceutically acceptable excipient or carrier. The excipients are added to the formulation for a variety of purposes.

In one embodiment, the present disclosure relates to solid or liquid formulation. In one embodiment, a liquid formulation can be for intravenous administration. In one embodiment, a solid formulation can comprise a lyophilized therapeutically active ingredient.

Diluents may be added to the formulations of the present invention. Diluents increase the bulk of a solid pharmaceutical composition, and may make a pharmaceutical dosage form containing the composition easier for the patient and care giver to handle. Diluents for solid compositions include, for example, microcrystalline cellulose (e.g., AVICEL), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT(r)), potassium chloride, powdered cellulose, sodium chloride, sorbitol, and talc. Diluents for liquid compositions include, but are not limited to, water, aqueous solutions of saccharides and/or sugar alcohols (e.g., glucose solution, dextrose solution, lactose solution, maltose solution, fructose solution), saline solution, and other aqueous medium.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g., carbopol), carboxymethylcellulose sodium, dextrin, ethyl cellulose, gelatin, guar gum, gum tragacanth, hydrogenated vegetable oil, hydroxyethyl cellulose, hydroxypropyl cellulose (e.g., KLUCEL), hydroxypropyl methyl cellulose (e.g., METHOCEL), liquid glucose, magnesium aluminum silicate, maltodextrin, methylcellulose, polymethacrylates, povidone (e.g., KOLLIDON, PLASDONE), pregelatinized starch, sodium alginate, and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g., AC-DI-SOL and PRIMELLOSE), colloidal silicon dioxide, croscarmellose sodium, crospovidone (e.g., KOLLIDON and POLYPLASDONE), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB), potato starch, and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

In some embodiments, the crystalline form of Compound I is maintained through the tableting process, including being under pressure from a punch and dye.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions may be prepared using the crystalline forms of the present invention and any other solid excipients where the components are dissolved or suspended in a liquid carrier such as water, vegetable oil, alcohol, polyethylene glycol, propylene glycol, or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as aspartame, lactose, sorbitol, saccharin, sodium saccharin, sucrose, aspartame, fructose, mannitol, and invert sugar may be added to improve the taste.

Preservatives and chelating agents such as alcohol, sodium benzoate, butylated hydroxyl toluene, butylated hydroxyanisole, and ethylenediamine tetraacetic acid may be added at levels safe for ingestion to improve storage stability.

A liquid composition may also contain a buffer such as gluconic acid, lactic acid, citric acid or acetic acid, sodium gluconate, sodium lactate, sodium citrate, or sodium acetate. Selection of excipients and the amounts used may be readily determined by the formulation scientist based upon experience and consideration of standard procedures and reference works in the field.

A liquid composition can be for injection. A liquid composition may contain sterile diluent, such as but not limited to, water, glucose solution, dextrose solution, sucrose solution, or saline solution.

In a liquid composition of the present disclosure, the pH of the composition can be adjusted using acidifying agent and/or alkalizing agent. In some embodiments, the pH of the composition can be adjusted with aqueous HCl and/or aqueous NaOH. In some embodiments, the pH of the composition is in the range from about 4.0 to about 6.0, including all values and subranges therebetween.

In some embodiments, the liquid composition is prepared under anaerobic conditions. In some embodiments, the materials used to prepare the liquid composition are sparged with nitrogen before use. In some embodiments, the liquid composition is sparged with nitrogen until soluble oxygen level reaches less than 1.0 ppm. In some embodiments, the liquid composition is prepared and sealed or capped under nitrogen.

In one embodiment, a liquid formulation comprises Compound I, or a pharmaceutically acceptable salt and/or solvate thereof, at a concentration greater than about 10 mg/mL, greater than about 11 mg/mL, greater than about 12 mg/mL, greater than about 13 mg/mL, greater than about 14 mg/mL, greater than about 15 mg/mL, greater than about 16 mg/mL, greater than about 17 mg/mL, greater than about 18 mg/mL, greater than about 19 mg/mL, greater than about 20 mg/mL, greater than about 21 mg/mL, greater than about 22 mg/mL, greater than about 23 mg/mL, greater than about 24 mg/mL, or greater than about 25 mg/mL, or any other value or range of values therein. In some embodiments, the formulation comprises Compound I, or a pharmaceutically acceptable salt and/or solvate thereof, at a concentration greater than about 15 mg/mL. In other embodiments, the formulation comprises Compound I, or a pharmaceutically acceptable salt and/or solvate thereof, at a concentration greater than about 25 mg/mL.

The solid compositions of the present invention include powders, granules, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and lozenges, as well as liquid syrups, suspensions, aerosols and elixirs.

The dosage form of the present invention may be a capsule containing the composition, preferably a powdered or granule solid composition of the invention, within either a hard or soft shell. The shell may be made from gelatin and optionally contain a plasticizer such as glycerin and sorbitol, and an opacifying agent or colorant.

A composition for tableting or capsule filling may be prepared by wet granulation. In wet granulation, some or all of the active ingredients and excipients in powder form are blended and then further mixed in the presence of a liquid, typically water that causes the powders to clump into granules. The granules are screened and/or milled, dried and then screened and/or milled to the desired particle size. The granules may be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granules that were described with reference to tableting; however, they are not subjected to a final tableting step.

In one embodiment, Compound I, or a pharmaceutically acceptable salt and/or solvate thereof, is reconstituted prior to administration in pharmaceutically acceptable carrier or solvent. In one embodiment, the reconstituted solution formulation comprising Compound I, or a pharmaceutically acceptable salt and/or solvate thereof, is administered by an IV.

In one embodiment, Compound I or a pharmaceutically acceptable salt and/or solvate thereof is provided in a lyophilized formulation. A lyophilized formulation can comprise bulking agents. In some embodiments, bulking agents can include, but are not limited to, sucrose, mannitol, and trehalose. See PCT/US2019/018225 for lyophilized formulation and liquid formulation of Compound I. The disclosures of PCT/US2019/018225 is hereby incorporated by reference in their entireties for all purposes. In other embodiments, a solid or liquid formulation is prepared using the lyophilized form of Compound I or a pharmaceutically acceptable salt and/or solvate thereof.

In one embodiment, a liquid formulation prepared from a lyophilized form of Compound I or a pharmaceutically acceptable salt and/or solvate thereof is provided. In one embodiment, a liquid formulation can be prepared from any form of Compound I or a pharmaceutically acceptable salt and/or solvate thereof is provided. In one embodiment, a liquid formulation can be prepared from any solid form of Compound I or a pharmaceutically acceptable salt and/or solvate thereof is provided. This liquid formulation can be used for IV administration.

The active ingredient and excipients may be formulated into compositions and dosage forms according to methods known in the art.

In one embodiment, a dosage form may be provided as a kit comprising crystalline form of Compound I and pharmaceutically acceptable excipients and carriers as separate components. In some embodiments, the dosage form kit allow physicians and patients to formulate an oral solution or injection solution prior to use by dissolving, suspending, or mixing the crystalline form of Compound I with pharmaceutically acceptable excipients and carriers. In one embodiment, a dosage form kit which provides crystalline form of Compound I has improved stability of Compound I compared to pre-formulated liquid formulations of Compound I.

A dosage form of the present invention may contain at least one of crystalline form of Compound I or a pharmaceutically acceptable salt or solvate thereof, as disclosed herein, in an amount of about 5 mg to about 500 mg, or any value in between. That is, a dosage form of the present invention may contain Compound I or a pharmaceutically acceptable salt or ester thereof, in an amount of about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 125 mg, 130 mg, 140 mg, 150 mg, 160 mg, 170 mg, 175 mg, 180 mg, 190 mg, 200 mg, 210 mg, 220 mg, 225 mg, 230 mg, 240 mg, 250 mg, 260 mg, 270 mg, 275 mg, 280 mg, 290 mg, 300 mg, 310 mg, 320 mg, 325 mg, 330 mg, 340 mg, 350 mg, 360 mg, 370 mg, 375 mg, 380 mg, 390 mg, 400 mg, 410 mg, 420 mg, 425 mg, 430 mg, 440 mg, 450 mg, 460 mg, 470 mg, 475 mg, 480 mg, 490 mg, or 500 mg.

A dosage form of the present invention may contain at least one of crystalline form of Compound I or a pharmaceutically acceptable salt or ester thereof, as disclosed herein, such that the total amount of Compound I (can be in various forms including free base, salts, polymorphs, etc) totals about 5 mg to about 500 mg, or any value in between. That is, a dosage form of the present invention comprise a crystalline form of Compound I or a pharmaceutically acceptable salt or ester thereof and optionally other forms of Compound I such that the total amount of Compound I is in an amount of about: 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 125 mg, 130 mg, 140 mg, 150 mg, 160 mg, 170 mg, 175 mg, 180 mg, 190 mg, 200 mg, 210 mg, 220 mg, 225 mg, 230 mg, 240 mg, 250 mg, 260 mg, 270 mg, 275 mg, 280 mg, 290 mg, 300 mg, 310 mg, 320 mg, 325 mg, 330 mg, 340 mg, 350 mg, 360 mg, 370 mg, 375 mg, 380 mg, 390 mg, 400 mg, 410 mg, 420 mg, 425 mg, 430 mg, 440 mg, 450 mg, 460 mg, 470 mg, 475 mg, 480 mg, 490 mg, or 500 mg.

In one embodiment, pharmaceutical formulations or compositions of the present invention contain 25-100% or 50-100% by weight, of at least one of crystalline form of Compound I as described herein, in the formulation or composition.

In one embodiment of the present disclosure, any one of the pharmaceutical formulation comprising Compound I or a pharmaceutically acceptable salt and/or solvate thereof comprises less than about 5% impurities. In some embodiments, the impurities are less than about 4%, less than about 3%, less than about 2%. In one embodiment, the impurities are less than about 1%.

In one embodiment of the present disclosure, any one of the pharmaceutical formulation comprising Compound I or a pharmaceutically acceptable salt and/or solvate thereof comprises less than 5% of impurities resulting from oxidation of Compound I or a pharmaceutically acceptable salt and/or solvate thereof. In one embodiment, the oxidation impurities are less than about 4%, less than about 3%, less than about 2% or about 1%. In one embodiment, the impurities are less than about 0.5% or less than about 0.1%.

In one embodiment, the oxidation product of Compound I is selected from:

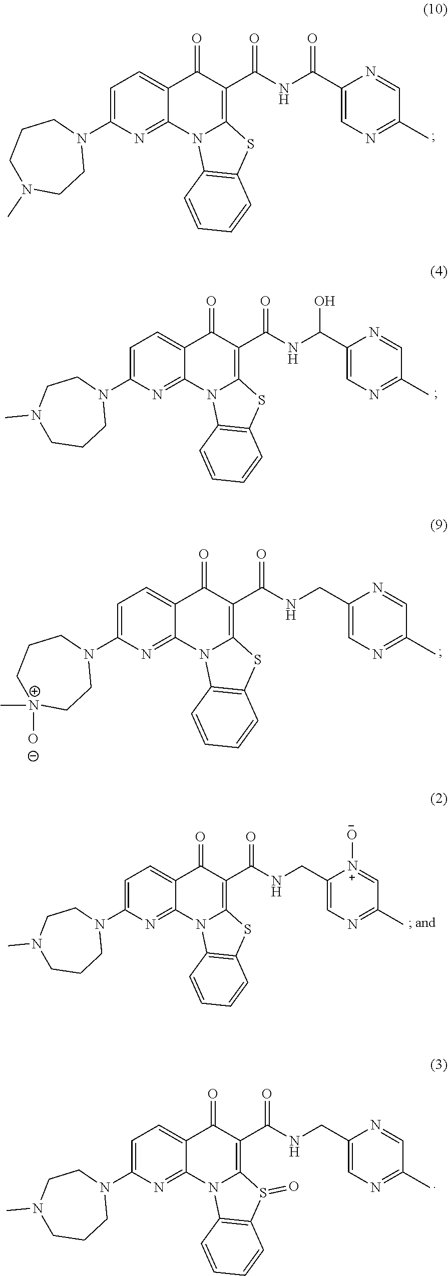

In one embodiment, the oxidation product is a ketone product. In some embodiments, the oxidation product is an N-oxide product. In one embodiment, the ketone product is Compound 10. In one embodiment, the N-oxide product is Compound 9.

In one embodiment of the present disclosure, any one of the pharmaceutical formulation comprising Compound I or a pharmaceutically acceptable salt and/or solvate thereof comprises less than about 0.5% or less than about 0.1% of Compound 9.

In one embodiment, the preparation of any one of the compositions, formulations, dosage forms as disclosed herein can be prepared under anaerobic conditions.

Therapeutic Use

The present invention also provides treatment of disorders related to proliferation of cells. In one aspect, there is provided a method for selectively activating p53 protein comprising contacting a cell afflicted by disorder related to cell proliferation with the present compound. In one embodiment, the method comprises contacting cancer and/or tumor cells with the crystalline form of Compound I, or a pharmaceutically acceptable salt, ester, and/or solvate thereof, as disclosed herein. In another embodiment, the method of contacting cancer and/or tumor cells with the crystalline form of Compound I, or a pharmaceutically acceptable salt, ester, and/or solvate thereof, as disclosed herein, may induce cell apoptosis or alleviate or prevent the progression of the disorder.

In one embodiment, the present invention provides a method for stabilizing G-quadruplex (G4) comprising contacting a cell afflicted by disorder related to cell proliferation with at least one compound of the invention. In one embodiment, the method comprises contacting cancer and/or tumor cells with at least one compound of the invention. In another embodiment, the method of contacting cancer and/or tumor cells with at least one compound of the present invention, may induce cell apoptosis or alleviate or delay the progression of the disorder.

In one embodiment, the compound of the present invention, can be administered in an amount effective to stabilize G4 in cancer and/or tumor cells, which may lead to cell death or apoptosis.

The present invention also provides methods of treating, preventing, ameliorating and/or alleviating the progression of disorders or conditions characterized by cell proliferation in a subject. More particularly, the methods of the present invention involve administration of an effective amount of the crystalline form of the quinolone compounds described herein, in a subject to treat a disorder or a condition characterized by cell proliferation. The crystalline form can be administered in an amount effective selectively activate p53 proteins in cancer and/or tumor cells, which may lead to cell death or apoptosis. The terms “subject” and “patient” are used interchangeably throughout the present application.

In one embodiment, the present invention relates to method of treating cancer comprising administering to a subject in need thereof an effective amount of the compound of the present invention. In one embodiment, cancer treated or ameliorated by the method as disclosed herein may be selected from Acute Lymphoblastic Leukemia, Acute Myeloid Leukemia, Adrenocortical Carcinoma, AIDS-Related Cancers, Kaposi Sarcoma, Lymphoma, Anal Cancer, Appendix Cancer, Astrocytomas, Childhood Atypical Teratoid/Rhabdoid Tumor, Basal Cell Carcinoma, Skin Cancer (Nonmelanoma), Childhood Bile Duct Cancer, Extrahepatic Bladder Cancer, Bone Cancer, Ewing Sarcoma Family of Tumors, Osteosarcoma and Malignant Fibrous Histiocytoma, Brain Stem Glioma, Brain Tumors, Embryonal Tumors, Germ Cell Tumors, Craniopharyngioma, Ependymoma, Bronchial Tumors, Burkitt Lymphoma (Non-Hodgkin Lymphoma), Carcinoid Tumor, Gastrointestinal Carcinoma of Unknown Primary, Cardiac (Heart) Tumors, Lymphoma, Primary, Cervical Cancer, Childhood Cancers, Chordoma, Chronic Lymphocytic Leukemia, Chronic Myelogenous Leukemia, Chronic Myeloproliferative Neoplasms Colon Cancer, Colorectal Cancer, Cutaneous T-Cell Lymphoma, Ductal Carcinoma In Situ, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Intraocular Melanoma, Retinoblastoma, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors, Extragonadal Cancer, Ovarian Cancer, Testicular Cancer, Gestational Trophoblastic Disease, Glioma, Brain Stem Cancer, Hairy Cell Leukemia, Head and Neck Cancer, Heart Cancer, Hepatocellular (Liver) Cancer, Histiocytosis, Langerhans Cell Cancer, Hodgkin Lymphoma, Hypopharyngeal Cancer, Intraocular Melanoma, Islet Cell Tumors, Pancreatic Neuroendocrine Tumors, Kaposi Sarcoma, Kidney Cancer, Renal Cell Cancer, Wilms Tumor and Other Childhood Kidney Tumors, Langerhans Cell Histiocytosis, Laryngeal Cancer, Leukemia, Chronic Lymphocytic Cancer, Chronic Myelogenous Cancer, Hairy Cell Cancer, Lip and Oral Cavity Cancer, Liver Cancer (Primary), Lobular Carcinoma In Situ (LCIS), Lung Cancer, Non-Small Cell Cancer, Small Cell Cancer, Lymphoma, Cutaneous T-Cell (Mycosis Fungoides and Sézary Syndrome), Hodgkin Cancer, Non-Hodgkin Cancer, Macroglobulinemia, Waldenström, Male Breast Cancer, Malignant Fibrous Histiocytoma of Bone and Osteosarcoma, Melanoma, Intraocular (Eye) Cancer, Merkel Cell Carcinoma, Mesothelioma, Malignant, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Chronic, Myeloid Leukemia, Acute, Myeloma Multiple, Chronic Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip and Oropharyngeal Cancer, Osteosarcoma and Malignant Fibrous Histiocytoma of Bone, Epithelial Cancer, Low Malignant Potential Tumor, Pancreatic Cancer, Pancreatic Neuroendocrine Tumors (Islet Cell Tumors), Papillomatosis, Paraganglioma, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Primary Central Nervous System Lymphoma, Rectal Cancer, Renal Cell (Kidney) Cancer, Retinoblastoma, Rhabdomyosarcoma, Salivary Gland Cancer, Sarcoma, Ewing Cancer, Kaposi Cancer, Osteosarcoma (Bone Cancer), Soft Tissue Cancer, Uterine Cancer, Sézary Syndrome, Skin Cancer, Childhood Melanoma, Merkel Cell Carcinoma, Nonmelanoma, Small Cell Lung Cancer, Small Intestine Cancer, Soft Tissue Sarcoma, Squamous Cell Carcinoma, Skin Cancer (Nonmelanoma), Childhood Squamous Neck Cancer with Occult Primary, Metastatic Cancer, Stomach (Gastric) Cancer, T-Cell Lymphoma, Cutaneous Cancer, Testicular Cancer, Throat Cancer, Thymoma and Thymic Carcinoma, Thyroid Cancer, Transitional Cell Cancer of the Renal Pelvis and Ureter, Unknown Primary, Carcinoma of Childhood, Unusual Cancers of Childhood, Urethral Cancer, Uterine Cancer, Endometrial Cancer, Uterine Sarcoma, Vaginal Cancer, Vulvar Cancer, Waldenström Macroglobulinemia, Wilms Tumor, or Women's Cancers.

Additionally, disclosed are methods for treating cancers, cancer cells, tumors, or tumor cells. Non limiting examples of cancer that may be treated by the methods of this disclosure include cancer or cancer cells of: large intestine, breast, lung, liver, pancreas, lymph node, colon, rectum, prostate, brain, head and neck, skin, ovary, cervical, thyroid, bladder, kidney, and blood and heart (e.g., leukemia, lymphoma, and carcinoma). Non limiting examples of tumors that may be treated by the methods of this disclosure include tumors and tumor cells of: large intestine, breast, lung, liver, pancreas, lymph node, colon, rectum, prostate, brain, head and neck, skin, kidney, and blood and heart (e.g., leukemia, lymphoma, and carcinoma), uterine, gastrointestine, larynx, and oral cavity.

In one embodiment, cancer treated or ameliorated by any one of the methods as disclosed herein may be selected from the group consisting of: heme cancer (hematologic malignancies), colorectal cancer, breast cancer, lung cancer, liver cancer, ovarian cancer, cervical cancer, Ewing's sarcoma, pancreatic cancer, cancer of the lymph nodes, colon cancer, prostate cancer, brain cancer, cancer of the head and neck, skin cancer, kidney cancer, cancer of the heart, uterine cancer, gastrointestinal malignancies, and carcinomas of the larynx and oral cavity. In some embodiments, the cancer treated or ameliorated by the method is selected from the group consisting of uterine cancer, gastrointestinal malignancies, and carcinomas of the larynx and oral cavity. In one embodiment, cancer treated or ameliorated by the method is hematologic malignancies which is selected from the group consisting of: leukemia, lymphoma, myeloma, and multiple myeloma. In one embodiment, cancer treated or ameliorated by any one of the methods as disclosed herein may be selected from the group consisting of: hematologic malignancies, colorectal cancer, breast cancer, lung cancer, liver cancer, ovarian cancer, cervical cancer, Ewing's sarcoma, pancreatic cancer, cancer of the lymph nodes, colon cancer, prostate cancer, brain cancer, cancer of the head and neck, skin cancer, kidney cancer, osteosarcoma, and cancer of the heart. In one embodiment, cancer treated or ameliorated by the method is heme cancer which is selected from the group consisting of: leukemia, lymphoma, myeloma, and multiple myeloma.

In one embodiment, the compound of the invention is useful for treating breast cancer. In one embodiment, the compound of the invention is useful for treating ovarian cancer. In one embodiment, the compound of the invention is useful for treating solid tumors. In one embodiment, the compound of the invention is useful for treating pancreatic cancer. In one embodiment, the compound of the invention is useful for treating pancreatic tumor. In one embodiment, the compound of the invention is useful for treating non-small cell lung cancer. In one embodiment, the compound of the invention is useful for treating hematologic malignancies. In one embodiment, the compound of the invention is useful for treating hematologic malignancies.

In one embodiment, cancer treated or ameliorated by any one of the methods as disclosed herein can be wherein the subject has a mutation in a DNA repair gene. In a specific embodiment, the DNA repair gene is a homologous recombinant gene. In another embodiment, the DNA repair gene is a gene in the homologous recombination (HR) dependent deoxyribonucleic acid (DNA) double strand break (DSB) repair pathway. In a specific embodiment, the DNA repair gene is a homologous recombinant (HR) or non-homologous end joining (NHEJ) gene. In another embodiment, the DNA repair gene is a gene in the homologous recombination (HR) or non-homologous end joining (NHEJ) dependent deoxyribonucleic acid (DNA) double strand break (DSB) repair pathway. In another method, the DNA repair gene is one or more genes selected from the group consisting of BRCA1, BRCA2, ATM, ATR, CHK1, CHK2, Rad51, RPA and XRCC3.

In one embodiment of any one of the methods as disclosed herein, the subject has a mutation in one or more genes in the HR pathway, Fanconi anemia pathway, mismatch repair pathway, ATM pathway, cell cycle pathway, p53 signaling pathway, polymerase pathway, topoisomerase pathway. In one embodiment, the subject has a mutation in one or more genes having a function in HR repair, ATM pathway, cell cycle, topoisomerase, double-strand break repair, excision repair, C-Myb transcription factor network, p-53 signaling, and/or apoptosis or genomic stability. In one embodiment, the subject has a mutation in one or more genes selected from BRCA, BRCA2, PTEN, ATM, CHEK1, TOP2A, ABL1, PER1, RAD51, ERCC5, NBN, TRIM28, SETMAR, RAD54L, EYA1, and TP53. In one embodiment, the subject has a mutation in one or more genes selected from ARID1A, ATM, ATR, BAP1, BARD1, BLM, BRCA1, BRCA2, CHEK1, CHEK2, ERCC3, FANCG, FANCI, FANCL, HELQ, MLHI, MRE11A, MSH2, MSH6, MUTYH, PMS1, POLE, POLR1B, PTEN, RAD17, RAD51D, RAD54L, TOP3A, and/or WRN.

In one embodiment, the subject has a mutation in one or more genes selected from BRCA1, BRCA2, TP53, and PALB2. In another embodiment, the subject has a mutation in BRCA1, and/or BRCA2 genes, and/or other genes of the HR pathway. In some embodiments, the mutation is a somatic mutation. In other embodiments, the mutation is a germline mutation.

In one embodiment, Compound I or a pharmaceutically acceptable salt thereof's efficacy is associated with a mutation or a copy number loss of a gene in the HR pathway or the Fanconi anemia pathway, wherein the gene is selected from: ARID1A, ATM, ATR, BAP1, BARD1, BLM, BRCA1, BRCA2, FANCG, FANCI, FANCL, HELQ, MRE1A, NBN, PALB2, PTEN, RAD51, RAD51D, RAD54L, and/or WRN. In one embodiment, Compound I or a pharmaceutically acceptable salt thereof's efficacy is associated with a mutation or a copy number loss of HR pathway gene BRCA2 and/or PALB2.

In another embodiment, cancer treated or ameliorated by the method comprises cancer cells harboring defects in BRCA1 gene (breast cancer type 1), BRCA2 (breast cancer type 2), and/or other members of the homologous recombination pathway. In another embodiment, the cancer cells are deficient in BRCA1 and/or BRCA2. In another embodiment, the cancer cells are homozygous for a mutation in BRCA1 and/or BRCA2. In another embodiment, the cancer cells are heterozygous for a mutation in BRCA1 and/or BRCA2. In some embodiments, the cancer cells are deficient in germline BRCA1 and/or BRCA2. In another embodiment, the cancer cells are deficient in somatic BRCA1 and/or BRCA2.

In one embodiment, cancer treated or ameliorated by any one of the methods as disclosed herein is BRCA2 deficient. In another embodiment, Compound I or a pharmaceutically acceptable salt or solvate thereof or the compound of the present invention induces more apoptotic cell death in BRCA2 deficient or BRCA2 knockout cells relative to BRCA2 proficient or BRCA2 wild type cells. In one embodiment, Compound I or a pharmaceutically acceptable salt or solvate thereof or the compound of the present invention is selectively toxic to BRCA2 deficient or BRCA2 knockout cells over BRCA2 proficient or BRCA2 wild type cells. In other embodiments, BRCA2 deficient or BRCA2 knockout cells exhibit higher sensitivity to Compound I or a pharmaceutically acceptable salt or solvate thereof or the compound of the present invention as compared to BRCA2 proficient or BRCA2 wild type cells.

In some embodiments, cancer treated or ameliorated by any one of the methods as disclosed herein is characterized by one or more mutations in the BRCA1 or BRCA2 genes. BRCA1 and BRCA2 are tumor suppressor genes, and encode proteins involved in DNA damage repair. Mutations that alter expression or activity of the BRCA1 or BRCA2 proteins may lead to the accumulation of genetic alterations in a cell, and can lead to cancer in a subject. Such mutations are referred to herein as “disease-associated mutations.” In some embodiments, the cancer is characterized one or more mutations in BRCA1 and BRCA2 genes. In some embodiments, the cancer is characterized one or more mutations in BRCA1 gene but has no mutations in BRCA2 gene. In some embodiments, the cancer is characterized one or more mutations in BRCA2 gene but has no mutations in BRCA1 gene.

In some embodiments, cancer treated or ameliorated by any one of the methods as disclosed herein is characterized by one or more disease-associated mutations in BRCA1 or BRCA2. In some embodiments, cancer is characterized by one or more disease-associated mutations in BRCA1 and BRCA2. In some embodiments, cancer is characterized by one or more disease-associated mutations in BRCA1 but harbors no disease-associated mutations in BRCA2. In some embodiments, cancer is characterized by one or more disease-associated mutations in BRCA2 but harbors no disease-associated mutations in BRCA1.

In one embodiment, cancer treated or ameliorated by any one of the methods as disclosed herein is BRCA mutant or BRCA-like mutant cancer. In some embodiments, the BRCA mutant or BRCA-like mutant cancer is a BRCA2-mutated cancer. In other embodiments, the BRCA mutant or BRCA-like mutant cancer is breast cancer, ovarian cancer, pancreatic cancer, or prostate cancer. In one embodiment, the BRCA mutant or BRCA-like mutant cancer is breast cancer or prostate cancer. In one embodiment, cancer treated or ameliorated by any one of the methods as disclosed herein is BRCA mutant cancer. In some embodiments, the BRCA mutant cancer is a BRCA2-mutated cancer. In other embodiments, the BRCA mutant cancer is breast cancer, ovarian cancer, pancreatic cancer, or prostate cancer. In other embodiments, the BRCA mutant cancer is breast cancer, ovarian cancer, or pancreatic cancer. In one embodiment, the BRCA mutant cancer is breast cancer or prostate cancer.

In some embodiments, cancer treated or ameliorated by any one of the methods as disclosed herein is BRCA-driven cancer. In some embodiments, cancer is BRCA1-driven cancer. In some embodiments, cancer is BRCA2-driven cancer. In some embodiments cancer is BRCA1- and BRCA2-driven cancer. In some embodiments, cancer is neither BRCA1- nor BRCA2-driven cancer.

In one embodiment, the present disclosure relates to methods for treating or ameliorating cell proliferation disorder in a human subject, comprising administering to a subject in need thereof a therapeutically effective amount of a compound of the invention or a formulation prepared from a compound of the present invention as disclosed herein. In some embodiments, the human subject carries a BRCA mutation. In other embodiments, the human subject carries a BRCA2 mutation. In another embodiment, the human subject is homozygous for a mutation in BRCA2.

In one embodiment, the present disclosure relates to methods for treating or ameliorating cell proliferation disorder in a human subject, comprising administering to a subject in need thereof a therapeutically effective amount of a compound of the invention or a formulation prepared from a compound of the present invention. In some embodiments, the human subject carries a BRCA mutation. In other embodiments, the human subject carries a BRCA2 mutation. In another embodiment, the human subject is homozygous for a mutation in BRCA2.

In one embodiment, the BRCA2 mutation is substitution, deleterious truncating, splicing, insertion or deletion of BRCA2 gene. In some embodiments, the BRCA2 mutation is a loss-of-function mutation.

In one embodiment, BRCA2 mutation exists as a coding change or mutation in one or more of 4088insA, c.68-80insT, c.793+34T>G, 999del5, 6503delTT, 4486delG, 2594delC, 5382insC, 3829delT, Q563X, 3438G>T, 1675delA, 999del5, 8295T4A, 9900insA, 5579insA, 7647delTG, 7253delAA, 9303ins31, 3034del4bp, 5910C3G, 6676insTA, 6085G>T, 8765delAG, 3398delAAAAG, 1499insA, 7525_7526insT, 6174delT, c.289G>T, c.2950G>T, c.7963C>T, c.8878C>T, IVS6p1G4A, 6503-6504delTT, 9132delC, 9254del5, c.9254_9258delATCAT, c.3492_3493insT, 9475A>G, c.9026_9030delATCAT, c.3264insT, c.8978_8991del14, c.156_157insAlu, 6238ins2del21, 10323delCins11, 8876delC, 8138_8142del5, c.8765_8766delAG, exons 21-24 del, c.6589delA, 4817A>G, 8477delAGA, 8984delG, G4X, 3783del10, c.5101C>T, c.5433_5436delGGAA, c.7806-2A>G, c.5291C>G, c.3975_3978dupTGCT, IVS16-2A>G, c.3318C>A, c.4790C>A, 9326insA and 6174delT, 8984delG, 1913T>A, 1342C>A, 3199A>G, 1093A>C, c.3394C>T, c.7697T>C, 5531delTT, C5507G, 6174delT, c.5373_5376 del GTAT, c.373G>T, S2219X, C1290Y, 6633del5, 3034delACAA, 818delA, exons 8-9 del, c.3036_3039delACAA, c.6024_6025_delTA, c.2732_2733insA, c.3870_3873delG, 4150G>T, 6027del4, c.5114_5117delTAAA, c.2639_2640delTG, 6880 insG, 3034 del AAAC, 695insT, 1528del4, 9318del4, S1099X, 5802delAATT, 8732C>A, c.2835C>A, c.7480C>T, 1627A.T, 3972delTGAG, 7708C.T, 7883delTTAA, c.2808_2811delACAA, c.3109C>T, c.7436_7805del370, c.9097_9098insA, 2670delC, 3073delT, 6696-7delTC, exons 4-11 dup, 4859delA, 4265delCT, 1342C.A, 490 delCT, 3337C>T, 5057delTG, g.-1235G>A, g.-26G>A, g.681+56C>T, c.865A>C, c.1114A>C, c.1365A>G, c.2229T>C, c.2971A>G, c.3396A>G, c.3516G>A, c.3807T>C, c.4415_4418delAGAA, c.5529A>C, c.6033_6034insGT, c.7242A>G, g.7435+53C>T, g.7806-14T>C, g.8755-66T>C, c.4415-4418delAGAA, c.6033insGT, c.5576_5579delTTAA, c.9485-1G>A, 4265delCT, 4859delA, 6775G>T, p.Gu2183X, c.2699_2704delTAAATG, 4706delAAAG, R2336P, IVS2+1G>A, 8765delAG, 999 del 5, 1537 del4, 5909 insA, c.211dupA, c.3381delT/3609delT, c.7110delA/7338delA, c.7235insG/7463insG, c.2826_2829del, c.6447_6448dup, c.5771_5774del, and/or 5999del4. See Karami, F. et al. BioMed Res. Int'l. 2013, 2013, Article ID 928562, which is hereby incorporated by reference in its entirety for all purposes.

In one embodiment, BRCA2 mutation exists as a coding change or mutation in one or more of c.8537_8538del AG, c.8537_8538del AG mutation in exon 20, c.859G>C, c. 859G>C in exon 7, c.4614T>C, p.Ser1538Ser synonymous mutation, c.5946delT, p.S1982fs, c.6819DelinsGT, c.6592G>T, c.3847_3848delGT, c.6821G>T, or c.6821G>T in exon 11.

In one embodiment, the compound of the present disclosure demonstrate sensitivity to a BRCA2 null cell line relative to the parental cell line. In one embodiment, the sensitivity of the BRCA2 null cell line is at least two hundred fold greater than the BRCA2 wild type cell line. In other embodiments, the sensitivity is at least twenty fold higher. In some embodiments, the sensitivity is at least 200 fold higher. In other embodiments, the sensitivity is at least 2, 5, 10, 15, 20, 25, 30, 35, 40, 45, 50, 60, 70, 80, 90, 100, 125, 150, 175, 200 or 400 fold higher.

In one embodiment, the present disclosure relates to methods for treating cancer in a subject, comprising administering a therapeutically effective amount of Compound I, or a pharmaceutically acceptable salt and/or solvate thereof to the subject, wherein the subject has a PALB2 mutation and/or a BRCA2 mutation. In one embodiment, the subject has a PALB2 mutation. In one embodiment, the subject has a BRCA2 mutation. In one embodiment, the subject has a PALB2 mutation and a BRCA2 mutation. In one embodiment, the subject has one or more additional gene mutation in the homologous recombination pathway.

In another embodiment, cancer treated or ameliorated by the method comprises cancer cells harboring defects in PALB2 gene. In another embodiment, the cancer cells are deficient in PALB2. In another embodiment, the cancer cells are homozygous for a mutation in PALB2. In another embodiment, the cancer cells are heterozygous for a mutation in PALB2.

In one embodiment, Compound I or a pharmaceutically acceptable salt or solvate thereof or the compound of the present invention induces more apoptotic cell death in PALB2 deficient or PALB2 knockout cells relative to PALB2 proficient or PALB2 wild type cells. In one embodiment, Compound I or a pharmaceutically acceptable salt or solvate thereof or the compound of the present invention is selectively toxic to PALB2 deficient or PALB2 knockout cells over PALB2 proficient or PALB2 wild type cells. In other embodiments, PALB2 deficient or PALB2 knockout cells exhibit higher sensitivity to Compound I or a pharmaceutically acceptable salt or solvate thereof or the compound of the present invention as compared to PALB2 proficient or PALB2 wild type cells.

In some embodiments, cancer treated or ameliorated by any one of the methods as disclosed herein is characterized by one or more mutations in the PALB2 genes. Mutations that alter expression or activity of the PALB2 proteins may lead to the accumulation of genetic alterations in a cell, and can lead to cancer in a subject. Such mutations are referred to herein as “disease-associated mutations.” In some embodiments, the cancer is characterized one or more mutations in PALB2 genes.

In some embodiments, cancer treated or ameliorated by any one of the methods as disclosed herein is characterized by one or more disease-associated mutations in PALB2.

In one embodiment, cancer treated or ameliorated by any one of the methods as disclosed herein is PALB2 mutant or PALB2-like mutant cancer. In some embodiments, the PALB2 mutant or PALB2-like mutant cancer is a PALB2-mutated cancer. In other embodiments, the PALB2 mutant or PALB2-like mutant cancer is breast cancer, ovarian cancer, pancreatic cancer, or prostate cancer. In one embodiment, the PALB2 mutant or PALB2-like mutant cancer is breast cancer or prostate cancer. In one embodiment, cancer treated or ameliorated by any one of the methods as disclosed herein is PALB2 mutant cancer (PALB2-mutated cancer). In other embodiments, the PALB2 mutant cancer is breast cancer, ovarian cancer, pancreatic cancer, or prostate cancer. In other embodiments, the PALB2 mutant cancer is breast cancer, ovarian cancer, or pancreatic cancer. In one embodiment, the PALB2 mutant cancer is breast cancer or prostate cancer.

In one embodiment, the PALB2 mutation is a loss-of-function mutation of the PALB2 gene. In one embodiment, the PALB2 mutation causes PALB2 gene to lose its function. In one embodiment, the PALB2 mutation is substitution, deleterious truncating, splicing, insertion or deletion of PALB2 gene. In some embodiments, the PALB2 mutation is a monoallelic loss-of-function mutation. In other embodiments, the PALB2 mutation is a biallelic loss-of-function mutation.

In one embodiment, the present disclosure relates to a method for treating or ameliorating cell proliferation disorder in a human subject, comprising administering to a subject in need thereof a therapeutically effective amount of a compound of the invention or a formulation prepared from a compound of the present invention as disclosed herein. In some embodiments, the human subject carries a PALB2 mutation. In another embodiment, the human subject is homozygous for a mutation in PALB2.

In some embodiments, cancer treated or ameliorated by any one of the methods as disclosed herein is PALB2-driven cancer.

In one embodiment, the present disclosure relates to a method for treating or ameliorating cell proliferation disorder in a human subject, comprising administering to a subject in need thereof a therapeutically effective amount of a compound of the invention or a formulation prepared from a compound of the present invention. In some embodiments, the human subject carries a PALB2 mutation. In another embodiment, the human subject is homozygous for a mutation in PALB2.

In one embodiment, PALB2 mutation exists as a coding change in one or more of c.48G>A, c.72del, c.156del, c.172_175del, c.196C>T, c.229del, c.451C>T, c.509_510del, c.757_758del, c.886del, c.956_962del, c.1027C>T, c.1037_1041del, c.1108C>T, c.1240C>T, c.1314del, c.1431del, c.1571C>G, c.1591_1600del, c.1592del, c.1653T>A, c.2074C>T, c.2167_2168del, c.2257C>T, c.2323C>T, c.2386G>T, c.2515-1G>T, c.2521del, c.2686dup, c.2718G>A, c.2787_2788del, c.2834+1G>T, c.2835-1G>C, c.2888del, c.2919_2920del, c.2982dup, c.3022del, c.3113G>A, c.3116del, c.3201+1G>C, c.3323del, c.3423_3426del, c.3426dup, c.3456dup, c.3497_3498del, c.3504_3505del, c.3549C>A, c.3549C>G, del5340 bp, or c.3362del. See Antoniou, A. C. et al. N. Engl. J. Med. 2014, 371, 497-506, which is hereby incorporated by reference in its entirety for all purposes.

In one embodiment, the present disclosure relates to methods for treating cancer in a subject, comprising a) determining if the subject harbors a BRCA1, BRCA2, or PALB2 mutation, and b) administering to a subject a therapeutically effective amount of a compound of the invention or a formulation prepared from a compound of the present invention. In one embodiment, the method of treating cancer in a subject comprises a) determining if the subject harbors a BRCA1, BRCA2, or PALB2 mutation, and b) administering to a subject a therapeutically effective amount of a compound of the invention or a formulation prepared from a compound of the present invention if the subject harbors a BRCA1, BRCA2, or PALB2 mutation. In one embodiment, the method of treating cancer in a subject comprises a) determining if the subject harbors BRCA1, BRCA2, or PALB2 mutation, and b) administering to a subject a therapeutically effective amount of a compound of the invention or a formulation prepared from a compound of the present invention if the subject harbors a BRCA2 or PALB2 mutation. In one embodiment, the method of treating cancer in a subject comprises a) determining if the subject harbors BRCA1, BRCA2, or PALB2 mutation, and b) administering to a subject a therapeutically effective amount of a compound of the invention or a formulation prepared from a compound of the present invention if the subject harbors a BRCA2 mutation. In one embodiment, the method of treating cancer in a subject comprises a) determining if the subject harbors BRCA1, BRCA2, or PALB2 mutation, and b) administering to a subject a therapeutically effective amount of a compound of the invention or a formulation prepared from a compound of the present invention if the subject harbors a PALB2 mutation.

In one embodiment, the present disclosure relates to methods for treating cancer in a subject, comprising a) determining if the subject harbors a disease-associated mutation in BRCA1, BRCA2, or PALB2 genes, and b) administering to a subject a therapeutically effective amount of a compound of the invention or a formulation prepared from a compound of the present invention. In one embodiment, the method of treating cancer in a subject comprises a) determining if the subject harbors a disease-associated mutation in BRCA1, BRCA2, or PALB2 genes, and b) administering to a subject a therapeutically effective amount of a compound of the invention or a formulation prepared from a compound of the present invention if the subject harbors a disease-associated mutation in BRCA1, BRCA2, or PALB2 genes. In one embodiment, the method of treating cancer in a subject comprises a) determining if the subject harbors a disease-associated mutation in BRCA1, BRCA2, or PALB2 genes, and b) administering to a subject a therapeutically effective amount of a compound of the invention or a formulation prepared from a compound of the present invention if the subject harbors a disease-associated mutation in BRCA2 or PALB2 genes. In one embodiment, the method of treating cancer in a subject comprises a) determining if the subject harbors a disease-associated mutation in BRCA1, BRCA2, or PALB2 genes, and b) administering to a subject a therapeutically effective amount of a compound of the invention or a formulation prepared from a compound of the present invention if the subject harbors a disease-associated mutation in BRCA2 gene. In one embodiment, the method of treating cancer in a subject comprises a) determining if the subject harbors a disease-associated mutation in BRCA1, BRCA2, or PALB2 genes, and b) administering to a subject a therapeutically effective amount of a compound of the invention or a formulation prepared from a compound of the present invention if the subject harbors a disease-associated mutation in PALB2 gene. In another embodiment, the cancer cells are deficient in BRCA1 and/or BRCA2. In another embodiment, the cancer cells are homozygous for a mutation in BRCA1 and/or BRCA2. In another embodiment, the cancer cells are heterozygous for a mutation in BRCA1 and/or BRCA2. In some embodiments, the cancer cells are deficient in germline BRCA1 and/or BRCA2. In another embodiment, the cancer cells are deficient in somatic BRCA1 and/or BRCA2.

Additionally, the present disclosure relates to methods for treating cancers, cancer cells, tumors, or tumor cells comprising administering a therapeutically effective amount of a compound of the invention or a formulation prepared from a compound of the present invention. The present disclosure also relates to methods for treating cancers, cancer cells, tumors, or tumor cells comprising administering a therapeutically effective amount of a compound of the invention or a formulation prepared from a compound of the present invention, to a subject in need thereof. Non limiting examples of cancer that may be treated by the methods of this disclosure include cancer or cancer cells of: large intestine, breast, ovary, cervix, lung, liver, pancreas, lymph node, colon, rectum, prostate, brain, head and neck, skin, kidney, osteosarcoma, bone (e.g., Ewing's sarcoma), blood and heart (e.g., leukemia, lymphoma, carcinoma), uterine, gastrointestinal malignancies, and carcinomas of the larynx and oral cavity. Non limiting examples of tumors that may be treated by the methods of this disclosure include tumors and tumor cells of: large intestine, breast, ovary, cervix, lung, liver, pancreas, lymph node, colon, rectum, prostate, brain, head and neck, skin, kidney, osteosarcoma, bone (e.g., Ewing's sarcoma), blood and heart (e.g., leukemia, lymphoma, carcinoma), uterine, gastrointestinal malignancies, and carcinomas of the larynx and oral cavity.

The present invention also provides methods of decreasing Pol I transcription comprising administering a compound of the invention or a formulation prepared from a compound of the present invention, to a subject in need. In some embodiments, the inhibition of Pol I transcription is in peripheral blood mononuclear cells (PBMC). In other embodiments, the inhibition of Pol I transcription can be observed in PBMC at one hour post-IV infusion of a dose comprising an effective amount of a compound of the invention or a formulation prepared from a compound of the present invention.

In one embodiment, the inhibition of Pol I transcription in PBMC 1 hour post-infusion is at an average level of about 15% inhibition or greater. In another embodiment, the Pol I transcription in PBMC 1 hour post-infusion is at an average level of about 5% inhibition or greater, about 10% inhibition or greater, about 15% inhibition or greater, about 20% inhibition or greater, about 25% inhibition or greater, about 30% inhibition or greater, about 35% inhibition or greater, about 40% inhibition or greater, about 45% inhibition or greater, about 50% inhibition or greater, about 55% inhibition or greater, about 65% inhibition or greater, or about 70% inhibition or greater.

In one embodiment of the present methods disclosed herein, the inhibition of Pol I transcription can be observed in MACS (magnetic-activated cell sorting) sorted tumor cells.

As used herein, administering can be effected or performed using any of the various methods known to those skilled in the art. A compound of the invention or a formulation prepared from a compound of the present invention, can be administered, for example, subcutaneously, intravenously, parenterally, intraperitoneally, intradermally, intramuscularly, topically, enteral (e.g., orally), rectally, nasally, buccally, sublingually, vaginally, by inhalation spray, by drug pump or via an implanted reservoir in dosage formulations containing conventional non-toxic, physiologically acceptable carriers or vehicles. A formulation or a composition comprising the compound of the present invention can be administered, for example, subcutaneously, intravenously, parenterally, intraperitoneally, intradermally, intramuscularly, topically, enteral (e.g., orally), rectally, nasally, buccally, sublingually, vaginally, by inhalation spray, by drug pump or via an implanted reservoir in dosage formulations containing conventional non-toxic, physiologically acceptable carriers or vehicles. In one embodiment, the composition of the present disclosure is administered intravenously.

Further, a compound of the invention or a formulation prepared from a compound of the present invention, can be administered to a localized area in need of treatment. For example, a formulation prepared from a compound of the present invention can be administered to a localized area in need of treatment. Administration to a localized area can be achieved by, for example, and not by way of limitation, local infusion during surgery, topical application, transdermal patches, by injection, by catheter, by suppository, or by implant (the implant can optionally be of a porous, non-porous, or gelatinous material), including membranes, such as sialastic membranes or fibers.

The formulation of a compound of the present invention for administration (e.g., syrup, elixir, capsule, tablet, foams, emulsion, gel, etc.) to a subject will depend in part on the route by which it is administered. For example, for mucosal (e.g., oral mucosa, rectal, intestinal mucosa, bronchial mucosa) administration, nose drops, aerosols, inhalants, nebulizers, eye drops or suppositories can be used. A compound of the invention or a formulation prepared from a compound of the present invention can also be used to coat bioimplantable materials to enhance neurite outgrowth, neural survival, or cellular interaction with the implant surface. A compound of the invention or a formulation prepared from a compound of the present invention can be administered together with other biologically active agents, such as anticancer agents, analgesics, anti-inflammatory agents, anesthetics and other agents which can control one or more symptoms or causes of a disorder or a condition characterized by cell proliferation.

In one embodiment, a compound of the invention or a formulation prepared from a compound of the present invention, as disclosed herein, can be administered in combination with one or more therapeutically active agent. In one embodiment, the one or more therapeutically active agent is an anticancer agent. In some embodiments, the one or more therapeutically active anticancer agents include, but are not limited to, paclitaxel, vinblastine, vincristine, etoposide, doxorubicin, hercepztin, lapatinib, gefitinib, erlotinib, tamoxifen, fulvestrant, anastrazole, lectrozole, exemestane, fadrozole, cyclophosphamide, taxotere, melphalan, chlorambucil, mechlorethamine, chlorambucil, phenylalanine, mustard, cyclophosphamide, ifosfamide, carmustine (BCNU), lomustine (CCNU), streptozotocin, busulfan, thiotepa, cisplatin, carboplatin, dactinomycin (actinomycin D), doxorubici(adriamycin), daunorubicin, idarubicin, mitoxantrone, plicamycin, mitomycin C, bleomycin, combinations thereof, and the like. In another embodiment, the one or more therapeutically active anticancer agents include, but are not limited to, PARP (poly (DP-ribose)polymerase) inhibitors. Suitable PARP inhibitors include, but are not limited to, 4-(3-(1-(cyclopropanecarbonyl)piperazine-4-carbonyl)-4-fluorobenzyl)phthalazin-1(2H)-one (Olaparib, AZD2281, Ku-0059436), 2-[(2R)-2-methylpyrrolidin-2-yl]-1H-benzimidazole-4-carboxamide (Veliparib, ABT-888), (8S,9R)-5-fluoro-8-(4-fluorophenyl)-9-(1-methyl-1H-1,2,4-triazol-5-yl)-8,9-dihydro-2H-pyrido[4,3,2-de]phthalazin-3(7H)-one(Talazoparib, BMN 673), 4-iodo-3-nitrobenzamide (Iniparib, BSI-201), 8-fluoro-5-(4-((methylamino)methyl)phenyl)-3,4-dihydro-2H-azepino[5,4,3-cd]indol-1(6H)-one phosphoric acid (Rucaparib, AG-014699, PF-01367338), 2-[4-[(dimethylamino)methyl]phenyl]-5,6-dihydroimidazo[4,5,1-jk][1,4]benzodiazepin-7(4H)-one (AG14361), 3-aminobenzamide (INO-1001), 2-(2-fluoro-4-((S)-pyrrolidin-2-yl)phenyl)-3H-benzo[d]imidazole-4-carboxamide (A-966492), N-(5,6-dihydro-6-oxo-2-phenanthridinyl)-2-acetamide hydrochloride (PJ34, PJ34 HCl), MK-4827, 3,4-dihydro-4-oxo-3,4-dihydro-4-oxo-N-[(1S)-1-phenylethyl]-2-quinazolinepropanamide (ME0328), 5-(2-oxo-2-phenylethoxy)-1(2H)-isoquinolinone (UPF-1069), 4-[[4-fluoro-3-[(4-methoxy-1-piperidinyl)carbonyl]phenyl]methyl]-1(2H)-phthalazinone (AZD 2461), 5-((3-chlorophenyl)amino)benzo[c][2,6]naphthyridine-8-carboxylic acid, and the like. In another embodiment, the one or more therapeutically active agent is an immunotherapeutic agent. In some embodiments, the one or more immunotherapeutic agents includes, but are not limited to, a monoclonal antibody, an immune effector cell, adoptive cell transfer, an immunotoxin, a vaccine, a cytokine, and the like.

In one embodiment, the one or more therapeutically active agent is selected from an alkylating agent, an anti-metabolite, a vinca alkaloid, a taxane, a topoisomerase inhibitor, an anti-tumor antibiotic, a tyrosine kinase inhibitor, an immunosuppressive macrolide, an Akt inhibitor, an HDAC inhibitor an Hsp90 inhibitor, an mTOR inhibitor, a PI3K/mTOR inhibitor, a PI3K inhibitor, a CDK (cyclin-dependent kinase) inhibitor, CHK (checkpoint kinase) inhibitor, PARP (poly (DP-ribose)polymerase) inhibitors, or combinations thereof.

In one embodiment, the one or more therapeutically active agent is a PI3K inhibitor. In another embodiment, the PI3K inhibitor is Idelalisib.

In one embodiment, the one or more therapeutically active agent is a PARP inhibitor. In another embodiment, the PARP inhibitor is Olaparib.

In other embodiments, the one or more therapeutically active agent is an agent that induces immune checkpoint blockade, such as PD-1 blockade and CTLA-4 blockade.

In some embodiments, the one or more therapeutically active agent is an antibody or an antigen-binding portion thereof that disrupts the interaction between Programmed Death-1 (PD-1) and Programmed Death Ligand-1 (PD-L1). In one embodiment, the one or more therapeutically active agent is selected from the group consisting of: an anti-PD-1 antibody, a PD-1 antagonist, an anti-PD-L1 antibody, a siRNA targeting expression of PD-1, a siRNA targeting the expression of PD-L1, and a peptide, fragment, dominant negative form, or soluble form of PD-1 or PD-L1.

In one embodiment, the one or more therapeutically active agent is a monoclonal antibody. In one embodiment, the monoclonal antibody is selected from the group consisting of anti-PD-1 antibody, nivolumab, pembrolizumab alemtuzumab, bevacizumab, brentuxima b vedotin, cetuximab, gemtuzumab ozogamicin, ibritumomab tiuxetan, ipilimumab, ofatumumab, panitumumab, rituximab, tositumomab, trastuzumab, anti-B7-H4, anti-B7-H1, anti-LAG3, BTLA, anti-Tim3, anti-B7-DC, anti-CD160, MR antagonist antibodies, anti-4-1BB, anti-OX40, anti-CD27, and/or CD40 agonist antibodies. In some embodiments, the one or more therapeutically active agent is an anti-PD-1 antibody. In other embodiments, an anti-PD-1 antibody is a humanized antibody. In one embodiment, the monoclonal antibody is selected from the group consisting of nivolumab and pembrolizumab. In a specific embodiment, the monoclonal antibody is nivolumab.

In some embodiments, one or more therapeutically active agent disclosed in WO 2017/087235 is hereby incorporated by reference in its entirety for all purposes.

In another embodiment, the crystalline form of Compound I, or a pharmaceutically acceptable salt, ester, and/or solvate of Compound I, as disclosed herein, can be administered in combination with radiotherapy.

Additionally, administration can comprise administering to the subject a plurality of dosages over a suitable period of time. Such administration regimens can be determined according to routine methods, upon a review of the instant disclosure.

Compound I or a pharmaceutically acceptable salt and/or solvate is generally administered in a dose of about 0.01 mg/kg/dose to about 100 mg/kg/dose. Alternately the dose can be from about 0.1 mg/kg/dose to about 10 mg/kg/dose; or about 1 mg/kg/dose to 10 mg/kg/dose. Time release preparations may be employed or the dose may be administered in as many divided doses as is convenient. When other methods are used (e.g. intravenous administration), crystalline forms are administered to the affected tissue at a rate from about 0.05 to about 10 mg/kg/hour, alternately from about 0.1 to about 1 mg/kg/hour. Such rates are easily maintained when these crystalline forms are intravenously administered as discussed herein. Generally, topically administered formulations are administered in a dose of about 0.5 mg/kg/dose to about 10 mg/kg/dose range. Alternately, topical formulations are administered at a dose of about 1 mg/kg/dose to about 7.5 mg/kg/dose or even about 1 mg/kg/dose to about 5 mg/kg/dose.

A range of from about 0.1 to about 100 mg/kg is appropriate for a single dose. Continuous administration is appropriate in the range of about 0.05 to about 10 mg/kg.

Drug doses can also be given in milligrams per square meter of body surface area rather than body weight, as this method achieves a good correlation to certain metabolic and excretionary functions. Moreover, body surface area can be used as a common denominator for drug dosage in adults and children as well as in different animal species (Freireich et al., (1966) Cancer Chemother Rep. 50, 219-244). Briefly, to express a mg/kg dose in any given species as the equivalent mg/sq m dose, the dosage is multiplied by the appropriate km factor. In an adult human, 100 mg/kg is equivalent to 100 mg/kg×37 kg/sq m=3700 mg/m².

A dosage form of the present invention may contain Compound I, or a pharmaceutically acceptable salt, ester, and/or solvate thereof, as disclosed herein, in an amount of about 5 mg to about 500 mg. That is, a dosage form of the present invention may contain Compound I in an amount of about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 125 mg, 130 mg, 140 mg, 150 mg, 160 mg, 170 mg, 175 mg, 180 mg, 190 mg, 200 mg, 210 mg, 220 mg, 225 mg, 230 mg, 240 mg, 250 mg, 260 mg, 270 mg, 275 mg, 280 mg, 290 mg, 300 mg, 310 mg, 320 mg, 325 mg, 330 mg, 340 mg, 350 mg, 360 mg, 370 mg, 375 mg, 380 mg, 390 mg, 400 mg, 410 mg, 420 mg, 425 mg, 430 mg, 440 mg, 450 mg, 460 mg, 470 mg, 475 mg, 480 mg, 490 mg, 500 mg or any value in between. In one embodiment, such dosage amount is administered to a patient as a daily dose either in a single dose or in divided portions served multiple times a day, such as twice, three times, or four times a day.

In one embodiment, compounds of the present invention or formulation prepared by compounds of the present invention are generally administered in a dose of about 1 mg of Compound I or a pharmaceutically acceptable salt and/or solvate thereof per body surface area of the subject (mg/m²) to about 2,000 mg/m², or any value or subranges therebetween, of Compound I, or a pharmaceutically acceptable salt and/or solvate thereof. In one embodiment, the compounds of the present invention or formulation prepared by compounds of the present invention are administered in a dose of about 25 mg/m² to about 2,000 mg/m², or any value or subranges therebetween, of Compound I, or a pharmaceutically acceptable salt and/or solvate thereof. In one embodiment, compounds of the present invention or formulation prepared by compounds of the present invention can be administered in a dose of about 25 mg/m², about 30 mg/m², about 35 mg/m², about 40 mg/m², about 45 mg/m², about 50 mg/m², about 55 mg/m², about 60 mg/m², about 65 mg/m², about 70 mg/m², about 75 mg/m², about 80 mg/m², about 85 mg/m², about 90 mg/m², about 95 mg/m², about 100 mg/m², about 110 mg/m², about 120 mg/m², about 125 mg/m², about 130 mg/m², about 140 mg/m², about 150 mg/m², about 160 mg/m², about 170 mg/m², about 175 mg/m², about 180 mg/m², about 190 mg/m², about 200 mg/m², about 210 mg/m², about 220 mg/m², about 225 mg/m², about 230 mg/m², about 240 mg/m², about 250 mg/m², about 260 mg/m², about 270 mg/m², about 275 mg/m², about 280 mg/m², about 290 mg/m², about 300 mg/m², about 310 mg/m², about 320 mg/m², about 325 mg/m², about 330 mg/m², about 340 mg/m², about 350 mg/m², about 360 mg/m², about 370 mg/m², about 375 mg/m², about 380 mg/m², about 390 mg/m², about 400 mg/m², about 410 mg/m², about 420 mg/m², about 425 mg/m², about 430 mg/m², about 440 mg/m², about 450 mg/m², about 460 mg/m², about 470 mg/m², about 475 mg/m², about 480 mg/m², about 490 mg/m², about 500 mg/m², about 510 mg/m², about 520 mg/m², about 525 mg/m², about 530 mg/m², about 540 mg/m², about 550 mg/m², about 560 mg/m², about 570 mg/m², about 575 mg/m², about 580 mg/m², about 590 mg/m², about 600 mg/m², about 610 mg/m², about 620 mg/m², about 625 mg/m², about 630 mg/m², about 640 mg/m², about 650 mg/m², about 660 mg/m², about 670 mg/m², about 675 mg/m², about 680 mg/m², about 690 mg/m², about 700 mg/m², about 710 mg/m², about 720 mg/m², about 725 mg/m², about 730 mg/m², about 740 mg/m², about 750 mg/m², about 760 mg/m², about 770 mg/m², about 775 mg/m², about 780 mg/m², about 790 mg/m², about 800 mg/m², about 810 mg/m², about 820 mg/m², about 825 mg/m², about 830 mg/m², about 840 mg/m², about 850 mg/m², about 860 mg/m², about 870 mg/m², about 875 mg/m², about 880 mg/m², about 890 mg/m², about 900 mg/m², about 910 mg/m², about 920 mg/m², about 925 mg/m², about 930 mg/m², about 940 mg/m², about 950 mg/m², about 960 mg/m², about 970 mg/m², about 975 mg/m², about 980 mg/m², about 990 mg/m², about 1000 mg/m², or any value in between, of Compound I, or a pharmaceutically acceptable salt and/or solvate thereof. In one embodiment, compounds of the present invention or formulation prepared by compounds of the present invention can be administered in a dose of about 50 mg, about 100 mg, about 150 mg, about 170 mg, about 325 mg, about 475 mg, or about 650 mg of Compound I or a pharmaceutically acceptable salt and/or solvate thereof. In some embodiments, the dose can vary depending on the health of the patients or the patient's sensitivity to Compound I, or a pharmaceutically acceptable salt and/or solvate thereof.

In one embodiment, compounds of the present invention or formulation prepared by compounds of the present invention are administered in a dose of about 50 mg/m² to about 800 mg/m², or any value or subranges therebetween, of Compound I, or a pharmaceutically acceptable salt and/or solvate thereof. In one embodiment, compounds of the present invention or formulation prepared by compounds of the present invention are administered in a dose of about 50 mg/m² to about 650 mg/m², or any value or subranges therebetween, of Compound I, or a pharmaceutically acceptable salt and/or solvate thereof. In one embodiment, compounds of the present invention or formulation prepared by compounds of the present invention are administered in a dose of about 100 mg/m² to about 700 mg/m², or any value or subranges therebetween, of Compound I, or a pharmaceutically acceptable salt and/or solvate thereof. In one embodiment, compounds of the present invention or formulation prepared by compounds of the present invention are administered in a dose of about 150 mg/m² to about 700 mg/m², or any value or subranges therebetween, of Compound I, or a pharmaceutically acceptable salt and/or solvate thereof. In one embodiment, compounds of the present invention or formulation prepared by compounds of the present invention are administered in a dose of about 150 mg/m² to about 650 mg/m², or any value or subranges therebetween, of Compound I, or a pharmaceutically acceptable salt and/or solvate thereof. In one embodiment, compounds of the present invention or formulation prepared by compounds of the present invention are administered in a dose of about 250 mg/m² to about 700 mg/m², or any value or subranges therebetween, of Compound I, or a pharmaceutically acceptable salt and/or solvate thereof. In one embodiment, compounds of the present invention or formulation prepared by compounds of the present invention are administered in a dose of about 300 mg/m² to about 700 mg/m², or any value or subranges therebetween, of Compound I, or a pharmaceutically acceptable salt and/or solvate thereof. In one embodiment, compounds of the present invention or formulation prepared by compounds of the present invention are administered in a dose of about 400 mg/m² to about 700 mg/m², or any value or subranges therebetween, of Compound I, or a pharmaceutically acceptable salt and/or solvate thereof. In one embodiment, compounds of the present invention or formulation prepared by compounds of the present invention are administered in a dose of about 425 mg/m² to about 675 mg/m², or any value or subranges therebetween, of Compound I, or a pharmaceutically acceptable salt and/or solvate thereof. In one embodiment, compounds of the present invention or formulation prepared by compounds of the present invention are administered in a dose of about 450 mg/m² to about 650 mg/m², or any value or subranges therebetween, of Compound I, or a pharmaceutically acceptable salt and/or solvate thereof. In one embodiment, compounds of the present invention or formulation prepared by compounds of the present invention are administered in a dose of about 475 mg/m² of Compound I, or a pharmaceutically acceptable salt and/or solvate thereof.

In one embodiment, compounds of the present invention or formulation prepared by compounds of the present invention are administered in a dose of about 150 mg/m² to about 300 mg/m², or any value or subranges therebetween, of Compound I, or a pharmaceutically acceptable salt and/or solvate thereof. In one embodiment, compounds of the present invention or formulation prepared by compounds of the present invention are administered in a dose of about 150 mg/m² to about 250 mg/m², or any value or subranges therebetween, of Compound I, or a pharmaceutically acceptable salt and/or solvate thereof. In one embodiment, compounds of the present invention or formulation prepared by compounds of the present invention are administered in a dose of about 170 mg/m² of Compound I, or a pharmaceutically acceptable salt and/or solvate thereof.

In one embodiment, compounds of the present invention or formulation prepared by compounds of the present invention can be generally administered in a dose of about less than about 500 mg/m² of Compound I, or a pharmaceutically acceptable salt and/or solvate thereof. In another embodiment, compounds of the present invention or formulation prepared by compounds of the present invention are generally administered in a dose of less than about 500 mg/m², less than about 490 mg/m², less than about 480 mg/m², less than about 475 mg/m², less than about 470 mg/m², less than about 460 mg/m², less than about 450 mg/m², less than about 440 mg/m², less than about 430 mg/m², less than about 420 mg/m², less than about 410 mg/m², less than about 400 mg/m², less than about 390 mg/m², less than about 380 mg/m², less than about 375 mg/m², less than about 370 mg/m², less than about 360 mg/m², less than about 350 mg/m², less than about 340 mg/m², less than about 330 mg/m², less than about 320 mg/m², less than about 310 mg/m², less than about 300 mg/m², less than about 290 mg/m², less than about 280 mg/m², less than about 275 mg/m², less than about 270 mg/m², less than about 260 mg/m², less than about 250 mg/m², less than about 240 mg/m², less than about 230 mg/m², less than about 220 mg/m², less than about 210 mg/m², less than about 200 mg/m², less than about 190 mg/m², less than about 180 mg/m², or less than about 170 mg/m², or any value in between, of Compound I, or a pharmaceutically acceptable salt and/or solvate thereof.

In some embodiments, compounds of the present invention or formulation prepared by compounds of the present invention can be administered to a cancer patient in a dose of less than about 750 mg/m², less than about 700 mg/m², less than about 600 mg/m², less than about 500 mg/m², less than about 475 mg/m², less than about 400 mg/m², less than about 325 mg/m², less than about 300 mg/m², less than about 200 mg/m², less than about 170 mg/m², or any subranges therein, of Compound I, or a pharmaceutically acceptable salt and/or solvate thereof. In other embodiments, compounds of the present invention or formulation prepared by compounds of the present invention can be administered to a cancer patient in a dose of less than about 170 mg/m² of Compound I, or a pharmaceutically acceptable salt and/or solvate thereof, every three weeks. In one embodiment, the cancer patient is a heme cancer patient.

In some embodiments, compounds of the present invention or formulation prepared by compounds of the present invention can be administered to a cancer patient in about 50 mg/m² to about 1,550 mg/m², about 150 mg/m² to about 1,250 mg/m², about 250 mg/m² to about 1,050 mg/m², about 350 mg/m² to about 950 mg/m², about 375 mg/m² to about 850 mg/m², about 425 mg/m² to about 850 mg/m², about 450 mg/m² to about 800 mg/m², or about 500 mg/m² to about 750 mg/m², or any subranges therein, of Compound I, or a pharmaceutically acceptable salt and/or solvate thereof. In some embodiments, compounds of the present invention or formulation prepared by compounds of the present invention can be administered to a cancer patient in a dose of less than about 750 mg/m² of Compound I, or a pharmaceutically acceptable salt and/or solvate thereof. In other embodiments, compounds of the present invention or formulation prepared by compounds of the present invention can be administered to a cancer patient in any of the dosing frequency, dosing cycle or dosing regimen described herein. In one embodiment, the treatment is for solid tumors.

In some embodiments, compounds of the present invention or formulation prepared by compounds of the present invention can be administered to a cancer patient at a dose of greater than about 50 mg/m² to provide clinical results including partial response, stable disease (no tumor growth), or tumor shrinkage. In some embodiments, compounds of the present invention or formulation prepared by compounds of the present invention can be administered to a cancer patient at a dose of greater than about 100 mg/m² to provide clinical results including partial response, stable disease, or tumor shrinkage. In some embodiments, compounds of the present invention or formulation prepared by compounds of the present invention can be administered to a cancer patient at a dose of greater than about 150 mg/m² to provide clinical results including partial response, stable disease, or tumor shrinkage.

A dosage form of the present invention may be administered, hourly, daily, weekly, or monthly. The dosage form of the present invention may be administered twice a day or once a day. The dosage form of the present invention may be administered with food or without food.

In one embodiment, compounds of the present invention or formulation prepared by compounds of the present invention, is administered once a week, once every two weeks, once every three weeks, once every four weeks, or once a month. In some embodiments, compounds of the present invention or formulation prepared by compounds of the present invention, is administered in a four-week treatment cycle comprising one administration weekly (QW×4). In some embodiments, compounds of the present invention or formulation prepared by compounds of the present invention, is administered in a four-week treatment cycle comprising one administration weekly for two weeks followed by two weeks of rest period (no treatment) (QW×2). In some embodiments, the administration is on a four-week treatment cycle comprising one administration weekly for three weeks followed by one week of rest period (no treatment). In some embodiments, compounds of the present invention or formulation prepared by compounds of the present invention, is administered in a three-week treatment cycle comprising one administration weekly for two weeks followed by one week of rest period. In another embodiment, compounds of the present invention or formulation prepared by compounds of the present invention, is administered once every three weeks. In other embodiments, compounds of the present invention or formulation prepared by compounds of the present invention, is administered once every three weeks by IV infusion.

In some embodiments, the treatment regimen with Compound I, or a pharmaceutically acceptable salt and/or solvate thereof, as disclosed herein, can last from 1 cycle to 20 cycles or greater period of time. An appropriate length of the treatment can be determined by a physician.

In some embodiments, the treatment with the compound of the invention results in PK ranges as disclosed in PCT/US2019/018225, the disclosures of which are hereby incorporated by reference in their entireties for all purposes.

Insofar as the crystalline forms disclosed herein can take the form of a mimetic or fragment thereof, it is to be appreciated that the potency, and therefore dosage of an effective amount can vary. However, one skilled in the art can readily assess the potency of a crystalline form of the type presently envisioned by the present application.

In settings of a gradually progressive disorder or condition characterized by cell proliferation, crystalline forms of the present application are generally administered on an ongoing basis. In certain settings administration of a crystalline form disclosed herein can commence prior to the development of disease symptoms as part of a strategy to delay or prevent the disease. In other settings a crystalline form disclosed herein is administered after the onset of disease symptoms as part of a strategy to slow or reverse the disease process and/or part of a strategy to improve cellular function and reduce symptoms.

It will be appreciated by one of skill in the art that dosage range will depend on the particular crystalline form, and its potency. The dosage range is understood to be large enough to produce the desired effect in which the neurodegenerative or other disorder and the symptoms associated therewith are ameliorated and/or survival of the cells is achieved, but not be so large as to cause unmanageable adverse side effects. It will be understood, however, that the specific dose level for any particular patient will depend on a variety of factors including the activity of the specific crystalline form employed; the age, body weight, general health, sex and diet of the individual being treated; the time and route of administration; the rate of excretion; other drugs which have previously been administered; and the severity of the particular disease undergoing therapy, as is well understood by those skilled in the art. The dosage can also be adjusted by the individual physician in the event of any complication. No unacceptable toxicological effects are expected when crystalline forms disclosed herein are used in accordance with the present application.

An effective amount of the crystalline forms disclosed herein comprise amounts sufficient to produce a measurable biological response. Actual dosage levels of active ingredients in a therapeutic crystalline form of the present application can be varied so as to administer an amount of the active crystalline form that is effective to achieve the desired therapeutic response for a particular subject and/or application. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

Further with respect to the methods of the present application, a preferred subject is a vertebrate subject. A preferred vertebrate is warm-blooded; a preferred warm-blooded vertebrate is a mammal. The subject treated by the presently disclosed methods is desirably a human, although it is to be understood that the principles of the present application indicate effectiveness with respect to all vertebrate species which are included in the term “subject.” In this context, a vertebrate is understood to be any vertebrate species in which treatment of a neurodegenerative disorder is desirable.

As such, the present application provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos or farms. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economical importance to humans. Thus, also provided are the treatment of livestock, including, but not limited to, domesticated swine, ruminants, ungulates, horses (including race horses), poultry, and the like.

The following examples further illustrate the present invention but should not be construed as in any way limiting its scope.

Any one of the formulations as disclosed herein can be used for any one of the methods disclosed herein, including treating cancer. Any one of the doses as disclosed herein for Compound I or a pharmaceutically acceptable salt and/or solvate thereof can be used for any one of the methods disclosed herein, including treating cancer. Any one of the dosing schedule as disclosed herein can be used for any one of the methods disclosed herein, including treating cancer.

Pharmacokinetics

In some embodiments, the treatment regimen with Compound I, or a pharmaceutically acceptable salt and/or solvate thereof, as disclosed herein, can last from 1 cycle to 20 cycles or greater period of time. One cycle can be one 4-week treatment (28 days). A 4-week treatment can be once a week administration for 3 weeks then 1 week off (Dosing Schedule A). An example of Dosing Schedule A is to administer Compound I or a pharmaceutically acceptable salt and/or solvate thereof on Days 1, 8 and 15 of the 28-day cycle. A 4-week treatment can be once a week administration for 2 weeks then 2 weeks off (Dosing Schedule B). An appropriate length of the treatment can be determined by a physician. An example of Dosing Schedule B is to administer Compound I or a pharmaceutically acceptable salt and/or solvate thereof on Days 1 and 8 of the 28-day cycle.

In one embodiment, Tₘₐₓ of Compound I, or a pharmaceutically acceptable salt and/or solvate thereof for human subjects who received a first dose of IV infusion administration at about 25 mg/m² to about 1,000 mg/m² of Compound I, or a pharmaceutically acceptable salt and/or solvate thereof is about 0.25 hr to about 1.25 hr, or any value or subranges therebetween. In another embodiment, Tₘₐₓ is about 0.5 hr to about 1.0 hr, or any value or subranges therebetween.

In one embodiment, Tₘₐₓ of Compound I for human subjects is about 0.3 hr to about 2.0 hr, or any value or subranges therebetween. In some embodiments, Tₘₐₓ is about 0.4 hr to about 1.5 hr, or any value or subranges therebetween. In one embodiment, the human subject receives Compound I or a pharmaceutically acceptable salt and/or solvate thereof in about 50 mg/m² to about 650 mg/m².

In one embodiment, Tₘₐₓ of Compound I for human subjects who received a first dose of IV infusion administration on Dosing Schedule A at about 325 mg/m² to about 650 mg/m² of Compound I, or a pharmaceutically acceptable salt and/or solvate thereof is about 0.4 hr to about 1.4 hr, or any value or subranges therebetween. In one embodiment, Tₘₐₓ is about 0.4 hr to about 1.1 hr, or any value or subranges therebetween. In one embodiment, Tₘₐₓ is about 0.6 hr to about 0.8 hr, or any value or subranges therebetween. In one embodiment, Tₘₐₓ is about 1.0 hr to about 1.4 hr, or any value or subranges therebetween. In one embodiment, Tₘₐₓ is about 1.1 hr to about 1.3 hr, or any value or subranges therebetween. In one embodiment, Tₘₐₓ is about 0.5 hr to about 1.1 hr, or any value or subranges therebetween. In one embodiment, Tₘₐₓ is about 0.7 hr to about 0.9 hr, or any value or subranges therebetween. In one embodiment, the dose at Dosing Schedule A is at about 325 mg/m², 475 mg/m², or 650 mg/m² of Compound I, or a pharmaceutically acceptable salt and/or solvate thereof.

In one embodiment, Tₘₐₓ of Compound I for human subjects who received a third dose of IV infusion administration on Dosing Schedule A at about 325 mg/m² to about 650 mg/m² of Compound I, or a pharmaceutically acceptable salt and/or solvate thereof is about 0.2 hr to about 1.2 hr, or any value or subranges therebetween. In one embodiment, Tₘₐₓ is about 0.4 hr to about 1.1 hr, or any value or subranges therebetween. In one embodiment, Tₘₐₓ is about 0.4 hr to about 1.1 hr, or any value or subranges therebetween. In one embodiment, Tₘₐₓ is about 0.7 hr to about 0.9 hr, or any value or subranges therebetween. In one embodiment, Tₘₐₓ is about 0.2 hr to about 1.3 hr, or any value or subranges therebetween. In one embodiment, Tₘₐₓ is about 0.9 hr to about 1.2 hr, or any value or subranges therebetween. In one embodiment, Tₘₐₓ is about 0.9 hr to about 1.1 hr, or any value or subranges therebetween. In one embodiment, the dose at Dosing Schedule A is at about 325 mg/m², 475 mg/m², or 650 mg/m² of Compound I, or a pharmaceutically acceptable salt and/or solvate thereof.

In one embodiment, Tₘₐₓ of Compound I for human subjects who received a first dose of IV infusion administration on Dosing Schedule B at about 50 mg/m² to about 475 mg/m² of Compound I, or a pharmaceutically acceptable salt and/or solvate thereof is about 0.4 hr to 1.6 hr, or any value or subranges therebetween. In one embodiment, Tₘₐₓ is about 0.4 hr to about 0.6 hr, or any value or subranges therebetween. In one embodiment, Tₘₐₓ is about 0.4 hr to about 0.8 hr, or any value or subranges therebetween. In one embodiment, Tₘₐₓ is about 0.5 hr to about 0.9 hr, or any value or subranges therebetween. In one embodiment, Tₘₐₓ is about 0.7 hr to about 0.9 hr, or any value or subranges therebetween. In one embodiment, Tₘₐₓ is about 0.9 hr to about 1.1 hr, or any value or subranges therebetween. In one embodiment, Tₘₐₓ is about 1.0 hr to about 1.2 hr, or any value or subranges therebetween. In one embodiment, the dose at Dosing Schedule B is at about 50 mg/m², 100 mg/m², 150 mg/m², 200 mg/m², 250 mg/m², 325 mg/m², or 475 mg/m², of Compound I, or a pharmaceutically acceptable salt and/or solvate thereof.

In one embodiment, Tₘₐₓ of Compound I for human subjects who received a second dose of IV infusion administration on Dosing Schedule B at about 50 mg/m² to about 475 mg/m² of Compound I, or a pharmaceutically acceptable salt and/or solvate thereof is about 0.1 hr to 2.7 hr, or any value or subranges therebetween. In one embodiment, Tₘₐₓ is about 0.2 hr to about 2.0 hr, or any value or subranges therebetween. In one embodiment, Tₘₐₓ is about 0.3 hr to about 1.5 hr, or any value or subranges therebetween. In one embodiment, Tₘₐₓ is about 0.4 hr to about 1.3 hr, or any value or subranges therebetween. In one embodiment, Tₘₐₓ is about 0.5 hr to about 1.2 hr, or any value or subranges therebetween. In one embodiment, the dose at Dosing Schedule B is at about 50 mg/m², 100 mg/m², 150 mg/m², 200 mg/m², 250 mg/m², 325 mg/m², or 475 mg/m², of Compound I, or a pharmaceutically acceptable salt and/or solvate thereof.

In one embodiment, the mean elimination half-life (T_{1/2}) of Compound I, or a pharmaceutically acceptable salt and/or solvate thereof for human subjects who received a first dose of IV infusion administration at about 25 mg/m² to about 1,000 mg/m² of Compound I, or a pharmaceutically acceptable salt and/or solvate thereof is about 20 hours to about 95 hours, or any value or subranges therebetween. In one embodiment, the mean elimination half-life (T_{1/2}) of Compound I, or a pharmaceutically acceptable salt and/or solvate thereof for human subjects who received a first dose of IV infusion administration at about 50 mg/m² to about 1,000 mg/m² of Compound I, or a pharmaceutically acceptable salt and/or solvate thereof is about 20 hours to about 50 hours, or any value or subranges therebetween.

In one embodiment, the mean elimination half-life (T_{1/2}) of Compound I, or a pharmaceutically acceptable salt and/or solvate thereof for human subjects is about 10 h to about 195 h, or any value or subranges therebetween. In one embodiment, mean T_{1/2} is about 20 h to about 125 h, or any value or subranges therebetween. In one embodiment, mean T_{1/2} is about 30 h to about 130 h, or any value or subranges therebetween. In one embodiment, mean T_{1/2} is about 30 h to about 100 h, or any value or subranges therebetween.

In one embodiment, mean Cₘₐₓ of Compound I for human subjects is about 200 ng/mL to about 6,000 ng/mL, or any value or subranges therebetween. In some embodiments, mean Cₘₐₓ is about 500 ng/mL to about 5,000 ng/mL, or any value or subranges therebetween. In one embodiment, the human subject receives Compound I or a pharmaceutically acceptable salt and/or solvate thereof in about 50 mg/m² to about 650 mg/m².

In one embodiment, mean Cₘₐₓ of Compound I for human subjects who received a first dose of IV infusion administration on Dosing Schedule A at about 325 mg/m² to about 650 mg/m² of Compound I, or a pharmaceutically acceptable salt and/or solvate thereof is about 800 ng/mL to about 4,500 ng/mL, or any value or subranges therebetween. In one embodiment, mean Cₘₐₓ is about 1,000 ng/mL to about 3,500 ng/mL, or any value or subranges therebetween. In one embodiment, mean Cₘₐₓ is about 800 ng/mL to about 1,500 ng/mL, or any value or subranges therebetween. In one embodiment, mean Cₘₐₓ is about 900 ng/mL to about 2,600 ng/mL, or any value or subranges therebetween. In one embodiment, mean Cₘₐₓ is about 1,200 ng/mL to about 2,300 ng/mL, or any value or subranges therebetween. In one embodiment, mean Cₘₐₓ is about 1,200 ng/mL to about 2,200 ng/mL, or any value or subranges therebetween. In one embodiment, mean Cₘₐₓ is about 2,000 ng/mL to about 4,100 ng/mL, or any value or subranges therebetween. In one embodiment, mean Cₘₐₓ is about 2,500 ng/mL to about 3,500 ng/mL, or any value or subranges therebetween. In one embodiment, the dose at Dosing Schedule A is at about 325 mg/m², 475 mg/m², or 650 mg/m² of Compound I, or a pharmaceutically acceptable salt and/or solvate thereof.

In one embodiment, mean Cₘₐₓ of Compound I for human subjects who received a third dose of IV infusion administration on Dosing Schedule A at about 325 mg/m² to about 650 mg/m² of Compound I, or a pharmaceutically acceptable salt and/or solvate thereof is about 500 ng/mL to about 6,000 ng/mL, or any value or subranges therebetween. In one embodiment, mean Cₘₐₓ is about 1,500 ng/mL to about 5,000 ng/mL, or any value or subranges therebetween. In one embodiment, mean Cₘₐₓ is about 500 ng/mL to about 5,500 ng/mL, or any value or subranges therebetween. In one embodiment, mean Cₘₐₓ is about 2,000 ng/mL to about 3,000 ng/mL, or any value or subranges therebetween. In one embodiment, mean Cₘₐₓ is about 2,500 ng/mL to about 6,000 ng/mL, or any value or subranges therebetween. In one embodiment, mean Cₘₐₓ is about 3,500 ng/mL to about 4,500 ng/mL, or any value or subranges therebetween. In one embodiment, the dose at Dosing Schedule A is at about 325 mg/m², 475 mg/m², or 650 mg/m² of Compound I, or a pharmaceutically acceptable salt and/or solvate thereof.

In one embodiment, mean Cₘₐₓ of Compound I for human subjects who received a first dose of IV infusion administration on Dosing Schedule B at about 50 mg/m² to about 475 mg/m² of Compound I, or a pharmaceutically acceptable salt and/or solvate thereof is about 400 ng/mL to about 2,800 ng/mL, or any value or subranges therebetween. In one embodiment, mean Cₘₐₓ is about 400 ng/mL to about 2,500 ng/mL, or any value or subranges therebetween. In one embodiment, mean Cₘₐₓ is about 500 ng/mL to about 2,100 ng/mL, or any value or subranges therebetween. In one embodiment, mean Cₘₐₓ is about 400 ng/mL to about 1,100 ng/mL, or any value or subranges therebetween. In one embodiment, mean Cₘₐₓ is about 600 ng/mL to about 1,600 ng/mL, or any value or subranges therebetween. In one embodiment, mean Cₘₐₓ is about 400 ng/mL to about 2,100 ng/mL, or any value or subranges therebetween. In one embodiment, mean Cₘₐₓ is about 550 ng/mL to about 1,300 ng/mL, or any value or subranges therebetween. In one embodiment, mean Cₘₐₓ is about 800 ng/mL to about 1,300 ng/mL, or any value or subranges therebetween. In one embodiment, mean Cₘₐₓ is about 1,400 ng/mL to about 2,500 ng/mL, or any value or subranges therebetween. In one embodiment, the dose at Dosing Schedule B is at about 50 mg/m², 100 mg/m², 150 mg/m², 200 mg/m², 250 mg/m², 325 mg/m², or 475 mg/m², of Compound I, or a pharmaceutically acceptable salt and/or solvate thereof.

In one embodiment, mean Cₘₐₓ of Compound I for human subjects who received a second dose of IV infusion administration on Dosing Schedule B at about 50 mg/m² to about 475 mg/m² of Compound I, or a pharmaceutically acceptable salt and/or solvate thereof is about 200 ng/mL to about 2,100 ng/mL, or any value or subranges therebetween. In one embodiment, mean Cₘₐₓ is about 400 ng/mL to about 1,900 ng/mL, or any value or subranges therebetween. In one embodiment, mean Cₘₐₓ is about 400 ng/mL to about 1,500 ng/mL, or any value or subranges therebetween. In one embodiment, mean Cₘₐₓ is about 200 ng/mL to about 1,100 ng/mL, or any value or subranges therebetween. In one embodiment, mean Cₘₐₓ is about 300 ng/mL to about 1,300 ng/mL, or any value or subranges therebetween. In one embodiment, mean Cₘₐₓ is about 700 ng/mL to about 1,800 ng/mL, or any value or subranges therebetween. In one embodiment, mean Cₘₐₓ is about 300 ng/mL to about 2,100 ng/mL, or any value or subranges therebetween. In one embodiment, mean Cₘₐₓ is about 200 ng/mL to about 1,500 ng/mL, or any value or subranges therebetween. In one embodiment, mean Cₘₐₓ is about 500 ng/mL to about 1,600 ng/mL, or any value or subranges therebetween. In one embodiment, mean Cₘₐₓ is about 1,400 ng/mL to about 2,000 ng/mL, or any value or subranges therebetween. In one embodiment, the dose at Dosing Schedule B is at about 50 mg/m², 100 mg/m², 150 mg/m², 200 mg/m², 250 mg/m², 325 mg/m², or 475 mg/m², of Compound I, or a pharmaceutically acceptable salt and/or solvate thereof.

In one embodiment, the unbound Compound I in area under the concentration-time curve for each dosing interval every 168 hours after steady-state exposures have been achieved (AUC_{ssτ}) is about 2 ng*hr/mL to about 300 ng*hr/mL, or any value or subranges therebetween, for subjects who received Compound I, or a pharmaceutically acceptable salt and/or solvate thereof in about 50 mg/m² to about 1,550 mg/m². In one embodiment, the AUC_{ssτ} is about 5 ng*hr/mL to about 200 ng*hr/mL, or any value or subranges therebetween, for subjects who received Compound I, or a pharmaceutically acceptable salt and/or solvate thereof in about 150 mg/m² to about 1,050 mg/m². In one embodiment, the AUC_{ssτ} is about 10 ng*hr/mL to about 150 ng*hr/mL, or any value or subranges therebetween, for subjects who received Compound I, or a pharmaceutically acceptable salt and/or solvate thereof in about 250 mg/m² to about 950 mg/m². In one embodiment, the AUC_{ssτ} is about 15 ng*hr/mL to about 140 ng*hr/mL, or any value or subranges therebetween, for subjects who received Compound I, or a pharmaceutically acceptable salt and/or solvate thereof in about 350 mg/m² to about 850 mg/m². In one embodiment, the AUC_{ssτ} is about 15 ng*hr/mL to about 150 ng*hr/mL, or any value or subranges therebetween, for subjects who received Compound I, or a pharmaceutically acceptable salt and/or solvate thereof in about 450 mg/m² to about 750 mg/m². In one embodiment, the AUC_{ssτ} is about 20 ng*hr/mL to about 120 ng*hr/mL, or any value or subranges therebetween, for subjects who received Compound I, or a pharmaceutically acceptable salt and/or solvate thereof in about 450 mg/m² to about 750 mg/m². In some embodiments, the subject is on QW×4 cycle dosing regimen, Dosing Schedule A or Dosing Schedule B.

In one embodiment, the AUC_{ssτ} is about 2 ng*hr/mL to about 250 ng*hr/mL for subjects who received Compound I, or a pharmaceutically acceptable salt and/or solvate thereof in about 50 mg/m² to about 1,550 mg/m². In one embodiment, the AUC_{ssτ} is about 5 ng*hr/mL to about 150 ng*hr/mL, or any value or subranges therebetween, for subjects who received Compound I, or a pharmaceutically acceptable salt and/or solvate thereof in about 150 mg/m² to about 1,050 mg/m². In one embodiment, the AUC_{ssτ} is about 10 ng*hr/mL to about 150 ng*hr/mL, or any value or subranges therebetween, for subjects who received Compound I, or a pharmaceutically acceptable salt and/or solvate thereof in about 250 mg/m² to about 950 mg/m². In one embodiment, the AUC_{ssτ} is about 15 ng*hr/mL to about 130 ng*hr/mL, or any value or subranges therebetween, for subjects who received Compound I, or a pharmaceutically acceptable salt and/or solvate thereof in about 350 mg/m² to about 850 mg/m². In one embodiment, the AUC_{ssτ} is about 15 ng*hr/mL to about 130 ng*hr/mL, or any value or subranges therebetween, for subjects who received Compound I, or a pharmaceutically acceptable salt and/or solvate thereof in about 450 mg/m² to about 750 mg/m². In one embodiment, the AUC_{ssτ} is about 20 ng*hr/mL to about 120 ng*hr/mL, or any value or subranges therebetween, for subjects who received Compound I, or a pharmaceutically acceptable salt and/or solvate thereof in about 450 mg/m² to about 750 mg/m². In some embodiments, the subject is on QW×2 four-week cycle dosing regimen.

In one embodiment, the AUC_{∞} (unbound Compound I in area under the concentration-time curve from time zero to infinity; total exposure) in a human subject (measured in plasma) is about 2,000 ng*hr/mL to about 110,000 ng*hr/mL, or any value or subranges therebetween, for subjects who received Compound I, or a pharmaceutically acceptable salt and/or solvate thereof in about 50 mg/m² to about 650 mg/m². In one embodiment, the AUC_{∞} is about 5,000 ng*hr/mL to about 70,000 ng*hr/mL, or any value or subranges therebetween.

In one embodiment, the AUC_{∞} in human subjects who received a first dose of IV infusion administration on Dosing Schedule A at about 325 mg/m² to about 650 mg/m² of Compound I, or a pharmaceutically acceptable salt and/or solvate thereof is about 10,000 ng*hr/mL to about 80,000 ng*hr/mL, or any value or subranges therebetween. In one embodiment, AUC_{∞} is about 24,000 ng*hr/mL to about 77,000 ng*hr/mL, or any value or subranges therebetween. In one embodiment, AUC_{∞} is about 25,000 ng*hr/mL to about 75,000 ng*hr/mL, or any value or subranges therebetween. In one embodiment, AUC_{∞} is about 15,000 ng*hr/mL to about 75,000 ng*hr/mL, or any value or subranges therebetween. In one embodiment, AUC_{∞} is about 24,000 ng*hr/mL to about 34,000 ng*hr/mL, or any value or subranges therebetween. In one embodiment, AUC_{∞} is about 28,000 ng*hr/mL to about 31,000 ng*hr/mL, or any value or subranges therebetween. In one embodiment, AUC_{∞} is about 10,000 ng*hr/mL to about 52,000 ng*hr/mL, or any value or subranges therebetween. In one embodiment, AUC_{∞} is about 11,000 ng*hr/mL to about 52,000 ng*hr/mL, or any value or subranges therebetween. In one embodiment, AUC_{∞} is about 30,000 ng*hr/mL to about 40,000 ng*hr/mL, or any value or subranges therebetween. In one embodiment, AUC_{∞} is about 38,000 ng*hr/mL to about 77,000 ng*hr/mL, or any value or subranges therebetween. In one embodiment, AUC_{∞} is about 50,000 ng*hr/mL to about 65,000 ng*hr/mL, or any value or subranges therebetween. In one embodiment, the dose at Dosing Schedule A is at about 325 mg/m², 475 mg/m², or 650 mg/m² of Compound I, or a pharmaceutically acceptable salt and/or solvate thereof.

In one embodiment, the AUC_{∞} in human subjects who received a third dose of IV infusion administration on Dosing Schedule A at about 325 mg/m² to about 650 mg/m² of Compound I, or a pharmaceutically acceptable salt and/or solvate thereof is about 12,000 ng*hr/mL to about 101,000 ng*hr/mL, or any value or subranges therebetween. In one embodiment, AUC_{∞} is about 15,000 ng*hr/mL to about 90,000 ng*hr/mL, or any value or subranges therebetween. In one embodiment, AUC_{∞} is about 12,000 ng*hr/mL to about 55,000 ng*hr/mL, or any value or subranges therebetween. In one embodiment, AUC_{∞} is about 20,000 ng*hr/mL to about 45,000 ng*hr/mL, or any value or subranges therebetween. In one embodiment, AUC_{∞} is about 24,000 ng*hr/mL to about 101,000 ng*hr/mL, or any value or subranges therebetween. In one embodiment, AUC_{∞} is about 45,000 ng*hr/mL to about 75,000 ng*hr/mL, or any value or subranges therebetween. In one embodiment, the dose at Dosing Schedule A is at about 325 mg/m², 475 mg/m², or 650 mg/m² of Compound I, or a pharmaceutically acceptable salt and/or solvate thereof.

In one embodiment, the AUC_{∞} in human subjects who received a first dose of IV infusion administration on Dosing Schedule B at about 50 mg/m² to about 475 mg/m² of Compound I, or a pharmaceutically acceptable salt and/or solvate thereof is about 5,00 ng*hr/mL to about 54,000 ng*hr/mL, or any value or subranges therebetween. In one embodiment, AUC_{∞} is about 5,500 ng*hr/mL to about 45,000 ng*hr/mL, or any value or subranges therebetween. In one embodiment, AUC_{∞} is about 5,500 ng*hr/mL to about 7,500 ng*hr/mL, or any value or subranges therebetween. In one embodiment, AUC_{∞} is about 10,000 ng*hr/mL to about 30,000 ng*hr/mL, or any value or subranges therebetween. In one embodiment, AUC_{∞} is about 15,000 ng*hr/mL to about 25,000 ng*hr/mL, or any value or subranges therebetween. In one embodiment, AUC_{∞} is about 7,000 ng*hr/mL to about 25,000 ng*hr/mL, or any value or subranges therebetween. In one embodiment, AUC_{∞} is about 18,000 ng*hr/mL to about 24,000 ng*hr/mL, or any value or subranges therebetween. In one embodiment, AUC_{∞} is about 13,500 ng*hr/mL to about 33,500 ng*hr/mL, or any value or subranges therebetween. In one embodiment, AUC_{∞} is about 19,000 ng*hr/mL to about 53,000 ng*hr/mL, or any value or subranges therebetween. In one embodiment, AUC_{∞} is about 30,000 ng*hr/mL to about 40,000 ng*hr/mL, or any value or subranges therebetween. In one embodiment, the dose at Dosing Schedule B is at about 50 mg/m², 100 mg/m², 150 mg/m², 200 mg/m², 250 mg/m², 325 mg/m², or 475 mg/m², of Compound I, or a pharmaceutically acceptable salt and/or solvate thereof.

In one embodiment, the AUC_{∞} in human subjects who received a second dose of IV infusion administration on Dosing Schedule B at about 50 mg/m² to about 475 mg/m² of Compound I, or a pharmaceutically acceptable salt and/or solvate thereof is about 1,000 ng*hr/mL to about 53,500 ng*hr/mL, or any value or subranges therebetween. In one embodiment, AUC_{∞} is about 2,500 ng*hr/mL to about 53,000 ng*hr/mL, or any value or subranges therebetween. In one embodiment, AUC_{∞} is about 8,500 ng*hr/mL to about 53,500 ng*hr/mL, or any value or subranges therebetween. In one embodiment, AUC_{∞} is about 8,500 ng*hr/mL to about 10,000 ng*hr/mL, or any value or subranges therebetween. In one embodiment, AUC_{∞} is about 15,000 ng*hr/mL to about 30,000 ng*hr/mL, or any value or subranges therebetween. In one embodiment, AUC_{∞} is about 15,000 ng*hr/mL to about 25,000 ng*hr/mL, or any value or subranges therebetween. In one embodiment, AUC_{∞} is about 6,000 ng*hr/mL to about 27,000 ng*hr/mL, or any value or subranges therebetween. In one embodiment, AUC_{∞} is about 10,000 ng*hr/mL to about 20,000 ng*hr/mL, or any value or subranges therebetween. In one embodiment, AUC_{∞} is about 24,000 ng*hr/mL to about 32,000 ng*hr/mL, or any value or subranges therebetween. In one embodiment, AUC_{∞} is about 19,000 ng*hr/mL to about 39,000 ng*hr/mL, or any value or subranges therebetween. In one embodiment, AUC_{∞} is about 25,000 ng*hr/mL to about 35,000 ng*hr/mL, or any value or subranges therebetween. In one embodiment, AUC_{∞} is about 19,000 ng*hr/mL to about 53,000 ng*hr/mL, or any value or subranges therebetween. In one embodiment, AUC_{∞} is about 30,000 ng*hr/mL to about 40,000 ng*hr/mL, or any value or subranges therebetween. In one embodiment, the dose at Dosing Schedule B is at about 50 mg/m², 100 mg/m², 150 mg/m², 200 mg/m², 250 mg/m², 325 mg/m², or 475 mg/m², of Compound I, or a pharmaceutically acceptable salt and/or solvate thereof.

### EXAMPLES

### Example 1: Synthesis of Compound 1

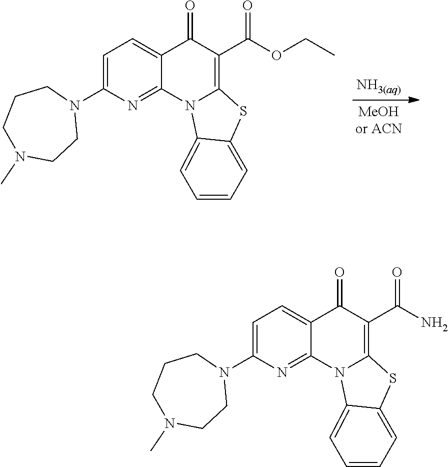

To a 5 L reactor was added ethyl 2-(4-methyl-1,4-diazepan-1-yl)-5-oxo-5H-benzo[4,5]thiazolo[3,2-a][1,8]naphthyridine-6-carboxylate (101 g, see WO 2009/046383 for synthesis) and acetonitrile (2 L). To the mixture was added 28-30% NH₃(aq) (1950 ml) then heated up to 60° C. (a lot of NH₃ gas released) with condenser temperature at −13° C. The mixture was stirred for 4 days and additional 28-30% NH3(aq) (400 ml) was added. The mixture was stirred for 2 days and additional 28-30% NH3(aq) (200 ml) was added. The mixture was stirred for 1 day and additional 28-30% NH3(aq) (100 ml) was added. After 10 days of stirring, a lot of solid precipitated. The mixture was cooled to room temperature and stirred for 1 day. The mixture was filtered to get 92 g of crude Compound 1 in 53.8% yield with 94.66% purity and the loss on drying (LOD) was 42.1%.

To a 2 L reactor was added 38 g of crude Compound 1 and 800 ml of acetonitrile. The resulting mixture was heated to 60° C. and then added 800 ml of 28-30% NH₃(aq). The mixture was stirred for 3 hr, filtrated at 50-60° C. and washed with 350 ml of acetonitrile. The wet cake was dried at 40° C. overnight to get 34.8 g of Compound 1(2-(4-methyl-1,4-diazepan-1-yl)-5-oxo-5H-benzo[4,5]thiazolo[3,2-a][1,8]naphthyridine-6-carboxamide) in 92% yield with 98.0% purity and the LOD was 2.30%. MS: m/z 408.136 [M+H]+.

### Biological Assays and Examples

### Example 2. Cell Viability Assessment and Cell Proliferation Assessment

The effect of Compound I on cell viability was assessed by Alamar Blue assay of metabolic activity in various cancer cell lines. Table 1 shows Compound I demonstrate broad spectrum antiproliferative activity in multiple cancer cell lines, while being significantly less active in normal cells.

TABLE 1
Compound I EC₅₀ in Cell Viability Assay

| Cell Line | Cancer Type | EC₅₀ (nM) |
|---|---|---|
| EOL-1 | Leukemia | 3 |
| SR | Leukemia | 5 |
| MOLT-3 | Leukemia | 6 |
| MV 4; 11 | Leukemia | 12 |
| SEM | Leukemia | 18 |
| A7 | Melanoma | 23 |
| NCI-H460 | Lung | 38 |
| THP-1 | Leukemia | 47 |
| NCI-H1299 | Lung | 55 |
| A375 | Melanoma | 58 |
| Jurkat | Leukemia | 64 |
| Ramos | Lymphoma | 66 |
| RPMI-8226 | Myeloma | 68 |
| NCI-H520 | Lung | 70 |
| MIA PaCa-2 | Pancreatic | 74 |
| SK-OV-3 | Ovarian | 78 |
| HL60 | Leukemia | 83 |
| MDA-MB-231 | Breast | 83 |
| BT-474 | Breast | 86 |
| COLO-205 | Colon | 96 |
| K562 | Leukemia | 104 |
| Hs 605.T | Breast | 116 |
| ZR-75-1 | Breast | 123 |
| Raji | Lymphoma | 133 |
| SKBr3 | Breast | 134 |
| MDA-MB-453 | Breast | 140 |
| Daudi | Lymphoma | 142 |
| HL60/MX2 | Leukemia | 147 |
| SK-MEL-24 | Melanoma | 147 |
| HCT-116 | Colon | 164 |
| NK92mi | Lymphoma | 165 |
| MDA-MB-468 | Breast | 171 |
| NCI-H2170 | Lung | 194 |
| U2OS | Osteosarcoma | 281 |
| BT-20 | Breast | 335 |
| MCF 7 | Breast | 347 |
| SUM 190PT | IBC* | 583 |
| BxPC-3 | Pancreatic | 664 |
| HT-29 | Colon | 741 |
| SUM 149PT | IBC* | 751 |
| PC-3 | Prostate | 1,100 |
| SK-MES-1 | Lung | 1,260 |
| Hs 578.T | Breast | 1,647 |
| UACC-812 | Breast | 1,830 |
| MDA-MB-361 | Breast | 2,100 |
| T47D | Breast | 2,337 |
| MDA-MB-175-VII | Breast | 2,780 |
| A549 | Lung | 4,900 |
| Saos-2 | Osteosarcoma | 5,000 |
| PANC-1 | Pancreatic | 5,000 |
| LNCaP | Prostate | 5,500 |
| CCD-1058Sk | Normal | 4,710 |
| CCD-1094Sk | Normal | 4,810 |
| CCD-1068Sk | Normal | 5,070 |
| BJ-hTERT | Normal | 5,174 |
| CCD-1096Sk | Normal | 5,260 |

*IBC = Invasive ductal breast carcinoma (inflammatory)

### Example 3. Pharmacokinetic (PK) Studies in Human

Pharmacokinetic studies and dose escalation studies were conducted in adult patients with metastatic, recurrent, locally, advanced, or unresectable solid malignancy (patients with histologically and/or cytologically confirmed solid malignancy), who received prior anti-cancer treatment(s) until disease progression, at 10 different dose levels of Compound I. Groups 1-7 were administered intravenously on days 1 and 8 of a 4-week cycle at 50, 100, 150, 200, 250, 325, and 475 mg/m² (lyophilized formulation), respectively. Groups 8-10 were administered intravenously on days 1,8, and 15 of a 4-week cycle at 325, 475, and 650 mg/m² (lyophilized formulation), respectively. Each patient received each dose over 1 hour by IV infusion on days described above.

Lyophilized Formulation:

2 L WFI was poured into a Schott bottled and degassed for 30 minutes with N₂ until dissolved oxygen content was ≤1 ppm (dissolved oxygen meter and probe; Mettler Toledo). 10% Mannitol solution was prepared by adding 150 g±1.0 g of mannitol and 1000 mL of degassed WFI to a tared 2 L Schott bottle containing a magnetic stirrer and wrapped in aluminum foil. To the Schott bottle containing mannitol, 15 mL of 2M HCl was added and stirred for approximately 10 minutes. The resulting solution was sparged with N₂ while stirring until dissolved oxygen content was ≤1 ppm (approx. 30 min). The headspace of the Schott bottle containing mannitol and HCl was purged with N₂ for 3 minutes and the bottle was sealed.

45.335 g of Compound I (free base) was weighed then added to the 2 L Schott bottle containing mannitol and HCl solution with continual magnetic stirring inside the Big Neat and CTS cabinets. After ˜55 minutes of stirring once Compound I was added, a suspension was observed. About 30 mL of 2M HCl was added and stirred for ˜25 minutes but the suspension persisted. About 5 mL of 2M HCl was added and stirred for ˜15 minutes then about 100 mL of degassed WFI was added and stirred for ˜5 minutes to provide a clear solution. Once a clear solution was obtained, the solution was sparged with N₂ while stirring until dissolved oxygen content was ≤1 ppm (approx. 40 min).

The pH of the Compound I solution (in Schott bottle) was measured then adjusted to pH 4.5±0.1 using 2 M HCl and 2 M NaOH solutions in a stepwise manner. After each addition of HCl or NaOH solutions, the solution was stirred for several minutes prior to verifying the pH. The pH adjusted Compound I solution was transferred to a 1000 mL volumetric flask and to a 500 mL volumetric flask and the flasks were filled with degassed WFI to the mark. Diluted solutions were transferred back to the original 2 L Schott bottle and sparged N₂ while stirring until dissolved oxygen content was ≤1 ppm (approx. 30 min). The headspace of the Schott bottle was purged with N₂ for 3 minutes and the bottle was sealed.

The final solution (pH adjusted and diluted) was filtered (chain including PVDF membrane filter 1×0.45 μm and 2×0.22 μm in series; peristaltic filtration pump) in the Big Neat and CTS cabinets and the bottle containing the filtered solution was sealed (The peristalitic pump filtration steps were not performed under N₂). The filtered solution was pumped (Watson Marlow Flexicon PF6-B pump; 100 RPM) into 30 mL Schott Clear Glass vials (5.0 mL filtered solution each). The glass vials were partially stoppered with 20 mm Freeze Dry Stopper Flurotec (West Pharma) one vial at a time with sterilized utensils inside a fume hood and placed on freeze dryer trays. The freeze dryer trays were transferred into freeze dry chamber and vial probe was positioned. Lyophilization was performed using the lyophilization cycle according to Table 3, Example 2. After lyophilization cycle finished, the vials were back filled with N₂ and the stopper was placed in situ, wiped down with isopropanol in the fume hood once removed from the freeze dryer, reconciled, then crimped.

They lyophilized formulation was reconstituted with 5% glucose in sterile water, or similar biologically acceptable fluid, for IV infusion.

Plasma Collection:

For patients in Groups 1-7, blood samples were typically collected at Day 1 at pre-infusion (t=−1 h), during infusion at about 30 minutes (t=−0.5 h), immediately post infusion (t=0 h), 30 min post-infusion, 1 h post-infusion, 2 h post-infusion, 4 h post-infusion, and 6 h post-infusion, as well as at about 24 h post-infusion, 48 h post-infusion, and 72 h post-infusion. On Day 8, blood samples were typically collected at pre-infusion (t=−1 h), during infusion at about 30 minutes (t=−0.5 h), immediately post infusion (t=0 h), 30 min post-infusion, 1 h post-infusion, 2 h post-infusion, 4 h post-infusion, and 6 h post-infusion, as well as at about 24 h post-infusion, and 168 h post-infusion (Day 15).

For patients in Groups 8-10, blood samples were typically collected at Day 1 at pre-infusion (t=−1 h), during infusion at about 30 minutes (t=−0.5 h), immediately post infusion (t=0 h), 30 min post-infusion, 1 h post-infusion, 2 h post-infusion, 4 h post-infusion, and 6 h post-infusion, as well as at about 24 h post-infusion, 48 h post-infusion, and 72 h post-infusion. On Day 8, blood samples were typically collected at pre-infusion (t=−1 h). On Day 15, blood samples were typically collected at pre-infusion (t=−1 h), during infusion at about 30 minutes (t=−0.5 h), immediately post infusion (t=0 h), 30 min post-infusion, 1 h post-infusion, 2 h post-infusion, 4 h post-infusion, and 6 h post-infusion, as well as at about 24 h post-infusion, and 168 h post-infusion (Day 22).

Levels of Compound I in patient's plasma were analyzed by liquid chromatography tandem mass spectrometry (LC/MS/MS) assay. Experimental procedures of the validated methods are summarized below. The pharmacokinetic parameters after first dose and second dose for Groups 1-7 and after first dose and third dose for Groups 8-10 are shown in Tables 2A-2J. In Tables 2A-2J: SD=standard deviation; Cₘₐₓ=maximum observed concentration; D=dose; Tₘₐₓ=time to maximum observed concentration; T_{1/2}=elimination half-life; Vd=volume of distribution; CL=drug clearance; and AUC_{∞}=area under the concentration-time curve from time zero to infinity (total exposure).

TABLE 2A
Summary of Mean Compound I Plasma PK Parameters
Following Day 1 (n = 4) and Day 8 (n =
3) dosed at 50 mg/m² [Group 1]

| | Dosed on Day 1 | | Dosed on Day 8 | |
|---|---|---|---|---|
| | Mean | SD | Mean | SD |
| Cₘₐₓ (ng/mL) | 728 | 291 | 642 | 385 |
| Cₘₐₓ/D (ng/mL/mg) | 8.14 | 3.14 | 7.23 | 4.98 |
| Tₘₐₓ (hr) | 0.758 | 0.298 | 1.01 | 0.861 |
| T_{1/2} (hr) | 60.1 | 50.5 | 42.6 | 22.2 |
| Vd (L) | 866 | 339 | 752 | 271 |
| CL (L/hr) | 15.9 | 9.40 | 16.5 | 12.6 |
| AUC_{∞} (ng*hr/mL) | 16300 | 24000 | 9120 | 7630 |
| AUC_{∞}/D (ng*hr/mL/mg) | 170 | 242 | 94.9 | 75.6 |

TABLE 2B
Summary of Mean Compound I Plasma PK Parameters Following
Day 1 and Day 8 dosed at 100 mg/m² [Group 2]

| | Dosed on Day 1 | | | Dosed on Day 8 | | |
|---|---|---|---|---|---|---|
| | n | Mean | SD | n | Mean | SD |
| Cₘₐₓ (ng/mL) | 4 | 1090 | 434 | 3 | 804 | 489 |
| Cₘₐₓ/D (ng/mL/mg) | 4 | 5.94 | 2.91 | 3 | 3.97 | 2.43 |
| Tₘₐₓ (hr) | 4 | 0.508 | 0.0167 | 3 | 0.700 | 0.346 |
| T_{1/2} (hr) | 3 | 37.3 | 8.30 | 3 | 60.4 | 9.13 |
| Vd (L) | 3 | 1400 | 59.0 | 3 | 1850 | 161 |
| CL (L/hr) | 3 | 9740 | 16800 | 3 | 6560 | 11300 |
| AUC_{∞} (ng*hr/mL) | 3 | 6650 | 803 | 3 | 9160 | 393 |
| AUC_{∞}/D (ng*hr/mL/mg) | 3 | 38.3 | 6.83 | 3 | 47.4 | 8.14 |

TABLE 2C
Summary of Mean Compound I Plasma PK Parameters
Following Day 1 (n = 4) and Day 8 (n = 4)
dosed at 150 mg/m² [Group 3]

| | Dosed on Day 1 | | Dosed on Day 8 | |
|---|---|---|---|---|
| | Mean | SD | Mean | SD |
| Cₘₐₓ (ng/mL) | 1230 | 807 | 1270 | 533 |
| Cₘₐₓ/D (ng/mL/mg) | 5.43 | 4.31 | 5.49 | 3.01 |
| Tₘₐₓ (hr) | 0.650 | 0.268 | 0.646 | 0.292 |
| T_{1/2} (hr) | 48.1 | 17.0 | 59.1 | 19.8 |
| Vd (L) | 900 | 240 | 939 | 116 |
| CL (L/hr) | 15.1 | 9.52 | 12.2 | 5.18 |
| AUC_{∞} (ng*hr/mL) | 20000 | 9750 | 21800 | 6890 |
| AUC_{∞}/D (ng*hr/mL/mg) | 84.5 | 41.9 | 91.1 | 29.1 |

TABLE 2D
Summary of Mean Compound I Plasma PK Parameters
Following Day 1 (n = 4) and Day 8 (n = 4)
dosed at 200 mg/m² [Group 4]

| | Dosed on Day 1 | | Dosed on Day 8 | |
|---|---|---|---|---|
| | Mean | SD | Mean | SD |
| Cₘₐₓ (ng/mL) | 8950 | 15100 | 1180 | 878 |
| Cₘₐₓ/D (ng/mL/mg) | 26.9 | 46.2 | 3.27 | 2.58 |
| Tₘₐₓ (hr) | 0.854 | 0.431 | 1.30 | 1.36 |
| T_{1/2} (hr) | 41.9 | 7.47 | 53.5 | 12.0 |
| Vd (L) | 1700 | 644 | 2050 | 733 |
| CL (L/hr) | 30.3 | 15.9 | 28.2 | 11.9 |
| AUC_{∞} (ng*hr/mL) | 15100 | 7860 | 16200 | 10200 |
| AUC_{∞}/D (ng*hr/mL/mg) | 42.7 | 25.8 | 44.5 | 29.2 |

TABLE 2E
Summary of Mean Compound I Plasma PK Parameters
Following Day 1 (n = 3) and Day 8 (n = 3)
dosed at 250 mg/m² [Group 5]

| | Dosed on Day 1 | | Dosed on Day 8 | |
|---|---|---|---|---|
| | Mean | SD | Mean | SD |
| Cₘₐₓ (ng/mL) | 1410 | 818 | 771 | 472 |
| Cₘₐₓ/D (ng/mL/mg) | 3.15 | 1.53 | 1.87 | 1.34 |
| Tₘₐₓ (hr) | 1.19 | 0.337 | 1.20 | 0.262 |
| T_{1/2} (hr) | 47.9 | 6.45 | 96.9 | 15.3 |
| Vd (L) | 1450 | 271 | 2160 | 320 |
| CL (L/hr) | 20.9 | 1.46 | 15.5 | 0.403 |
| AUC_{∞} (ng*hr/mL) | 20900 | 2800 | 28100 | 3630 |
| AUC_{∞}/D (ng*hr/mL/mg) | 48.0 | 3.46 | 64.6 | 1.70 |

TABLE 2F
Summary of Mean Compound I Plasma PK Parameters
Following Day 1 (n = 3) and Day 8 (n = 3)
dosed at 325 mg/m² [Group 6]

| | Dosed on Day 1 | | Dosed on Day 8 | |
|---|---|---|---|---|
| | Mean | SD | Mean | SD |
| Cₘₐₓ (ng/mL) | 1070 | 209 | 1040 | 497 |
| Cₘₐₓ/D (ng/mL/mg) | 2.14 | 0.380 | 2.09 | 1.01 |
| Tₘₐₓ (hr) | 1.02 | 0.0255 | 0.672 | 0.298 |
| T_{1/2} (hr) | 66.9 | 45.2 | 86.5 | 13.7 |
| Vd (L) | 1930 | 593 | 2220 | 323 |
| CL (L/hr) | 23.4 | 8.38 | 18.4 | 6.01 |
| AUC_{∞} (ng*hr/mL) | 23500 | 9440 | 29000 | 8980 |
| AUC_{∞}/D (ng*hr/mL/mg) | 46.9 | 17.5 | 57.8 | 16.1 |

TABLE 2G
Summary of Mean Compound I Plasma PK Parameters
Following Day 1 (n = 3) and Day 8 (n = 3)
dosed at 475 mg/m² [Group 7]

| | Dosed on Day 1 | | Dosed on Day 8 | |
|---|---|---|---|---|
| | Mean | SD | Mean | SD |
| Cₘₐₓ (ng/mL) | 1940 | 531 | 1710 | 221 |
| Cₘₐₓ/D (ng/mL/mg) | 2.48 | 1.00 | 2.15 | 0.568 |
| Tₘₐₓ (hr) | 0.861 | 0.314 | 0.678 | 0.308 |
| T_{1/2} (hr) | 81.8 | 41.2 | 73.8 | 40.0 |
| Vd (L) | 2590 | 397 | 2430 | 654 |
| CL (L/hr) | 25.1 | 10.1 | 28.9 | 19.1 |
| AUC_{∞} (ng*hr/mL) | 36200 | 16600 | 35400 | 16800 |
| AUC_{∞}/D (ng*hr/mL/mg) | 44.0 | 16.0 | 44.1 | 22.0 |

TABLE 2H
Summary of Mean Compound I Plasma PK Parameters Following
Day 1, Day 8, and Day 15 dosed at 325 mg/m² [Group 8]

| | Dosed on Day 1 | | | Dosed on Day 15 | | |
|---|---|---|---|---|---|---|
| | n | Mean | SD | n | Mean | SD |
| Cₘₐₓ (ng/mL) | 4 | 1140 | 295 | 4 | 21100 | 38000 |
| Cₘₐₓ/D (ng/mL/mg) | 4 | 2.04 | 0.589 | 4 | 35.5 | 63.2 |
| Tₘₐₓ (hr) | 4 | 0.767 | 0.289 | 4 | 0.783 | 0.319 |
| T_{1/2} (hr) | 3 | 83.4 | 5.63 | NA | NA | NA |
| Vd (L) | 3 | 2320 | 240 | NA | NA | NA |
| CL (L/hr) | 3 | 19.3 | 1.48 | NA | NA | NA |
| AUC_{∞} (ng*hr/mL) | 3 | 29100 | 4440 | NA | NA | NA |
| AUC_{∞}/D (ng*hr/mL/mg) | 3 | 52.1 | 4.19 | NA | NA | NA |

TABLE 2I
Summary of Mean Compound I Plasma PK Parameters Following
Day 1, Day 8, and Day 15 dosed at 475 mg/m² [Group 9]

| | Dosed on Day 1 | | | Dosed on Day 15 | | |
|---|---|---|---|---|---|---|
| | n | Mean | SD | n | Mean | SD |
| Cₘₐₓ (ng/mL) | 5 | 1750 | 801 | 5 | 2870 | 2310 |
| Cₘₐₓ/D (ng/mL/mg) | 5 | 2.01 | 0.876 | 5 | 3.34 | 2.79 |
| Tₘₐₓ (hr) | 5 | 1.18 | 0.169 | 5 | 0.737 | 0.492 |
| T_{1/2} (hr) | 5 | 61.5 | 15.5 | 4 | 59.0 | 11.2 |
| Vd (L) | 5 | 2320 | 869 | 4 | 2570 | 1040 |
| CL (L/hr) | 5 | 27.6 | 12.9 | 4 | 31.9 | 16.0 |
| AUC_{∞} (ng*hr/mL) | 5 | 36300 | 15000 | 4 | 32500 | 19600 |
| AUC_{∞}/D (ng*hr/mL/mg) | 5 | 42.7 | 17.8 | 4 | 39.8 | 24.5 |

TABLE 2J
Summary of Mean Compound I Plasma PK Parameters Following
Day 1 (n = 5), Day 8 and Day 15 (n = 5) dosed at 650
mg/m² [Group 10]

| | Dosed on Day 1 | | Dosed on Day 15 | |
|---|---|---|---|---|
| | Mean | SD | Mean | SD |
| Cₘₐₓ (ng/mL) | 3080 | 1020 | 4170 | 1570 |
| Cₘₐₓ/D (ng/mL/mg) | 2.61 | 1.11 | 3.44 | 1.34 |
| Tₘₐₓ (hr) | 0.810 | 0.279 | 1.05 | 0.0527 |
| T_{1/2} (hr) | 122 | 71.9 | 93.6 | 78.8 |
| Vd (L) | 3700 | 1710 | 2620 | 1100 |
| CL (L/hr) | 23500 | 8470 | 24900 | 12400 |
| AUC_{∞} (ng*hr/mL) | 57500 | 18700 | 62200 | 38200 |
| AUC_{∞}/D (ng*hr/mL/ | 46.5 | 14.1 | 48.6 | 23.8 |
| mg) | | | | |

As shown in Tables 2A-2G, following Day 1 and Day 8 doses from a Day 1/Day 8 Q4w regimen (Groups 1-7), Compound I generally exhibited a semi-logarithmic biphasic kinetic behavior on both days, with a steadily distribution phase followed by a long and slow elimination phase. This was similar across all dose levels. In addition, this was also observed at both Day 1 and Day 15 doses from a Day 1/Day 8/Day 15 Q4w regimen (Groups 8-10; Tables 2H-2J).

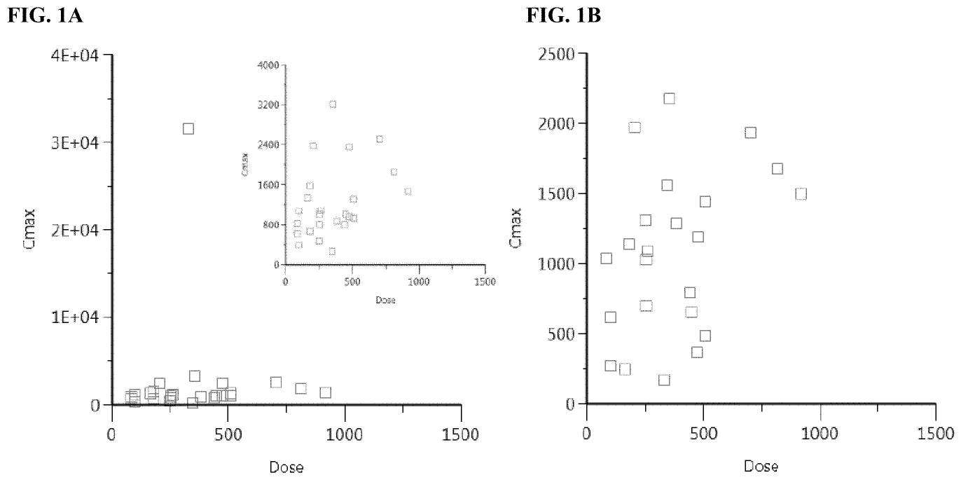

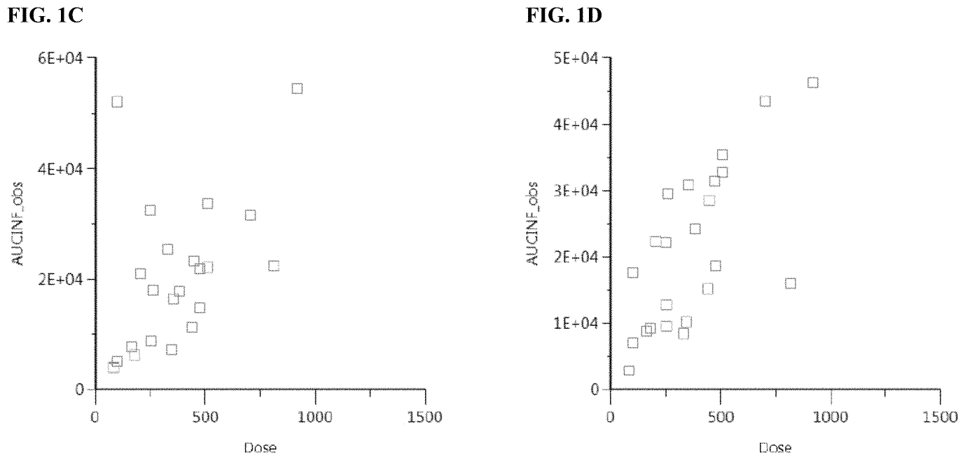

From the Day 1/Day 8 Q4w regimen (Groups 1-7), following Day 1 administration, Compound I was observed reaching maximal plasma concentrations at 0.508±0.0167 hr to 1.19±0.337 hr (Tₘₐₓ) across all dose levels tested (nominal 50 mg/m² to 475 mg/m²). Cₘₐₓ values ranged from 728±291 ng/mL to 1,940±531 ng/mL. There appeared to be an outlier at the dose of 200 mg/m² with the mean Cₘₐₓ value of 8,950±15,100 ng/mL. On Day 8, Cₘₐₓ were observed from 642±385 ng/mL to 1,710±221 ng/mL. Total exposures (AUC_{∞}) following both days were observed from 6,650±803 hr*ng/mL to 36,200±16,600 ng/mL and from 9,120±7,630 hr*ng/mL to 35,400±16,800 hr*ng/mL for Day 1 and Day 8 dosing, respectively. There appeared to be some correlation between dose and exposure across the 7 dose regimens tested, despite inter-subject PK variability (FIGS. 1A-1D). Apparent elimination half-lives were long and ranged from 37.3±8.3 hr to 81.8±41.2 hr following Day 1 dose and from 42.6±22.2 hr to 96.9±15.3 hr following Day 8 dose. There did not appear to be apparent differences observed between the two doses.

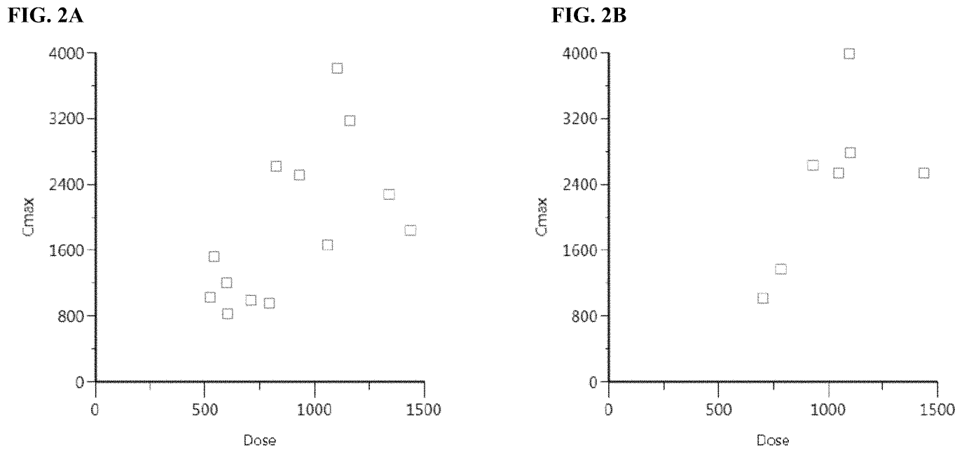

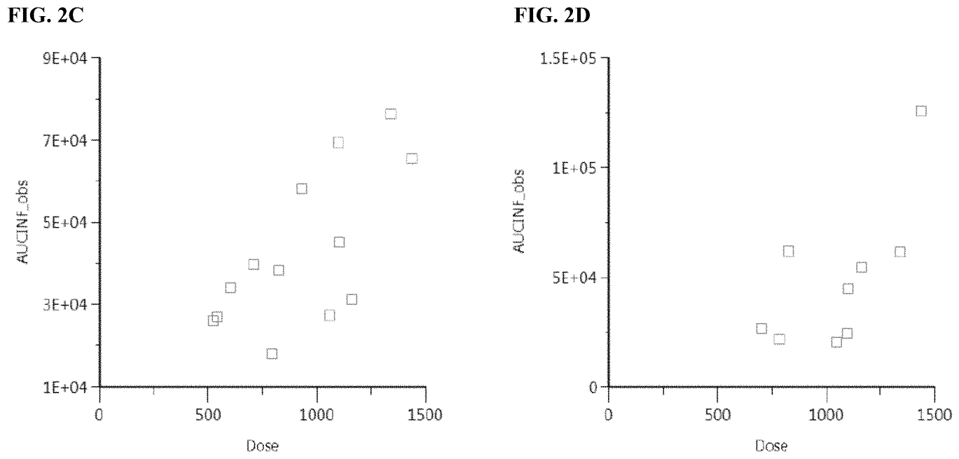

From the Day 1/Day 8/Day 15 Q4w regimen (Groups 8-10), following Day 1 administration, Compound I was observed reaching maximal plasma concentrations at 0.767±0.289 hr to 1.18±0.169 hr (Tₘₐₓ). Cₘₐₓ values ranged from 1,140±295 ng/mL to 3,080±1,020 ng/mL. On Day 15, Cₘₐₓ values ranged from 2,870±2,310 ng/mL to 4,170±1,570 ng/mL. There appeared to be an outlier at the dose of 325 mg/m² with the mean Cₘₐₓ value of 21,100±38,000 ng/mL. Total exposures (AUC_{∞}) following both days were observed from 29,100±4,440 hr*ng/mL to 57,500±18,700 ng/mL and from 32,500±19,600 hr*ng/mL to 62,200±38,200 hr*ng/mL for Day 1 and Day 15 dosing, respectively. There appeared to be some correlation between dose and exposure across the 3 dose regimens tested, despite inter-subject PK variability (FIGS. 2A-2D). Apparent elimination half-lives were long and ranged from 61.5±15.5 hr to 122±71.9 hr following Day 1 dose and from 59.0±11.2 hr to 93.6±78.8 hr following Day 15 dose. There did not appear to be apparent differences observed between the first and the third dose.

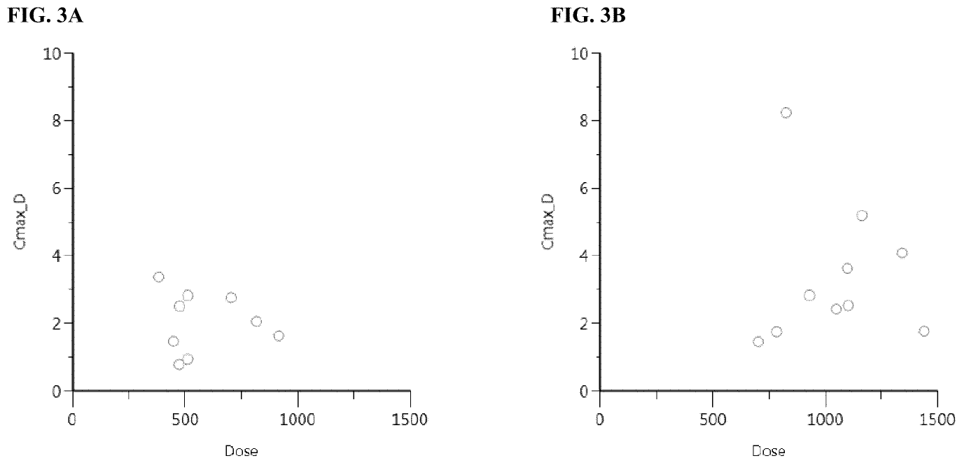

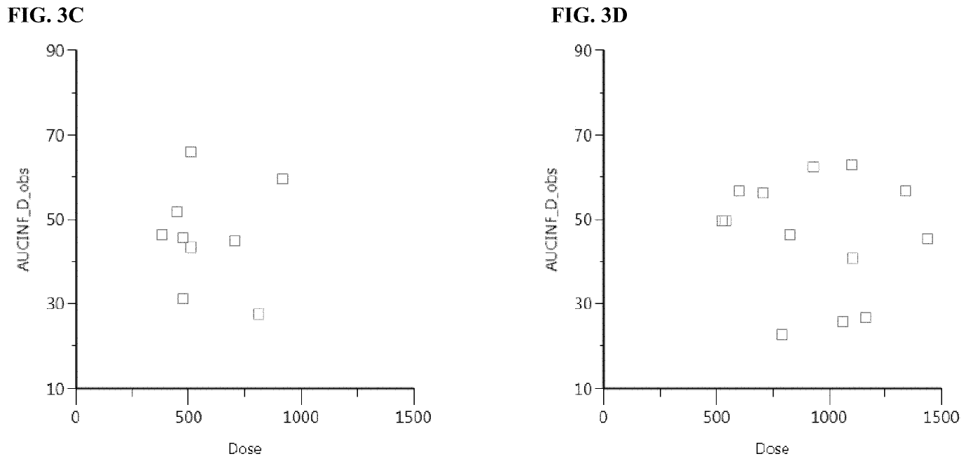

Between the two dose schedules (Groups 1-7 vs Groups 8-10), there were no apparent differences between exposure and elimination half-lives due to large inter-subject variability (FIGS. 3A-3D).

No dose level toxicity (DLT) were observed among Groups 1-10. There were 5 treatment-related non-DLT grade 3 photosensitivity events (in Groups 1, 5, and 8-10) that were reversible and were secondary to lack of photo-protective measures. Three serious adverse events (SAEs) were considered related to Compound I (photosensitivity of the skin (n=2) and photosensitivity of the eyes (n=1)). Treatment-related AEs≥10% were photosensitivity of the skin (59%), photosensitivity of the eyes (21%), mucositis (15%), nausea (44%), hand-foot syndrome (23%), headache (10%) and rash (10%).

From this PK studies, it was determined that 475 mg/m² is an appropriate dose for Day 1/Day 8/Day 15 Q4w regimen for Phase II studies.

Of the 40 patients treated, 34 have discontinued from the study either due to objective progressive disease (n=29), symptomatic progression of disease (n=4) or withdrawal of consent (n=1). In terms of best response, 4 patients, including 3 with breast cancer, have a confirmed partial response (2 germline BRCA2, 1 germline BRCA2 VUS, germline PALB2) and an additional 6 patients, including 2 with breast cancer, (4 germline BRCA2, 2 somatic BRCA1/2) with stable disease as best response for >4 cycles. Of the patients in Example 3, 8 patients who had received prior PARP inhibitor therapy all had early progression after ≤2 cycles but only 1 ovarian cancer patient and one metastatic breast cancer patient had BRCA2 mutations.

Assay of Human Plasma Samples

The concentrations of Compound I in human plasma samples collected from subjects of as discussed above. Compound I concentrations in plasma samples were measured based on 9 levels of calibration standards from 5.00 to 5000 ng/mL, and 3 levels of QC at 15.0 ng/mL (low), 2500 ng/mL (mid), and 4000 ng/mL (high).

LC/MS/MS Validated Method of Measuring Compound I in Human Plasma (Sodium Heparin)

Sample Preparation

Two reference standard master stock solutions of Compound I were prepared by dissolving the Compound 1 reference standard with 50:50:0.1 ACN:diH₂O:FA (v/v/v) and sonicating for 7 minutes (diH₂O=deionized water). The resulting solutions were further diluted with 50:50:0.1 ACN:diH₂O:FA (v/v/v) into a series of working stock solutions (WSS) for spiking of Compound I calibration standards and QC samples. The master stock solutions were prepared fresh on the day of assay, or stored at nominal 2° C. to 8° C. for later use. Master stock solutions were used within the stability timeframe.

The internal standard master stock solution of (2-(4-Methyl-piperazin-1-yl)-5-oxo-5H-7-thia-1,11b-diaza-benzo[c]fluorene-6-carboxylic acid (5-methyl-pyrazin-2-ylmethyl)-amide) (Compound A) was prepared by dissolving the Compound A internal standard in 50:50:0.1 ACN:diH₂O:FA (v/v/v). This stock solution was stored at nominal 2° C. to 8° C. prior to use. Stored internal standard stock solution was verified to be suitable for use by demonstrating the lack of interference to the detection of Compound I in each assay batch. On the day of assay, the stock solution was further diluted with 50:50:0.1 ACN:diH₂O:FA (v/v/v) into a working stock solution for spiking of samples.

To prepare each calibration standard and QC sample, 10 μL of appropriate working stock solution was added to PPE tubes. The blank assay matrix was centrifuged at 500×g for 1 minute to remove precipitate, and the centrifuged assay matrix was poured into a new, appropriately labelled container. An aliquot of 190 μL of centrifuged assay matrix was added to all tubes and vortex mixed. A volume of 10 μL of spiking ITS (internal standard) was added to all secondary PPE tubes, except for blk samples, and 100 μL of the appropriately spiked sample was transferred to the secondary PPE tubes and vortex mixed. All were protein precipitated through the addition of 600 μL of ACN, vortex mixed, and centrifuged at 13700×g for 5 minutes. The supernatant was transferred to 13×100 mm disposable glass tubes, and evaporated to dryness at 40° C. 2° C. under air or nitrogen flow. The dried sample was reconstituted by adding 50:50:0.1 ACN:diH2O:FA (v/v/v), vortex mixed for 1 minute, sonicated for 7 minutes, and centrifuged at 2440×g for 5 minutes. The sample is transferred to LC-vials for loading to the instrument.

Human plasma test samples were thawed unassisted at room temperature. Once thawed to room temperature, samples were vortex mixed at max speed. A volume of 10 μL of spiking ITS was added to PPE tubes, and 100 μL of test sample was added to the appropriate PPE tube. Test samples undergo the same process for protein precipitation, drying, and reconstitution as calibration and QC samples. For test samples requiring dilution, 50 μL of test sample was transferred to a secondary PPE tube, and 450 μL of blank assay matrix was added, and vortexed thoroughly. A total volume of 100 μL of diluted test sample is added to a PPE tube containing ITS for assay. The reconstituted sample will be transferred to a LC vial for LC/MS/MS analysis along with calibration standards and QC samples

LC/MS/MS Instrumentation Instrument Parameters

An Agilent Model 1100 HPLC system coupled with a Waters Micromass™ Quattro-LC or Quattro-Micro tandem triple quadrupole mass spectrometers controlled by Micromass MassLynx® Version 4.0 were used for the assay.

The analytical method parameters are summarized below:
Isocratic
Mobile Phase: elution using 7:3 (v/v) ACN: 75 mM ammonium formate (pH 2.5) with formic acid
Column: Zorbax 300-SCX, 3×50 mm, 5 μm
Flow rate: 0.5 mL/min
Run time: 6 min
Injection volume: 5 μL
MS mode: ESI positive MRM mode

Data collection and chromatographic peak integration were performed using Micromass MassLynx® Version 4.0. Regression analyses were performed by Thermo Watson™ Bioanalytical LIMS Version 7.4.0.0. Descriptive statistics were performed using Thermo Watson™ Bioanalytical LIMS Version 7.4.0.0 or Microsoft Excel 2007.

Non-compartmental pharmacokinetic parameters were derived from a well-established software program, Phoenix WinNonlin ver 8.

### Example 4: Evaluation of Predictive Biomarkers of Response to Compound I

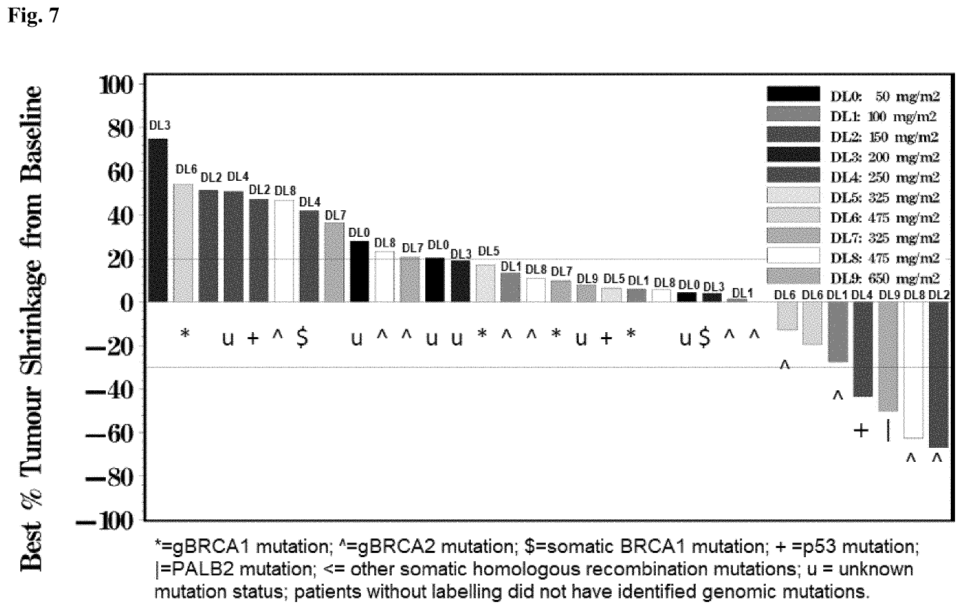

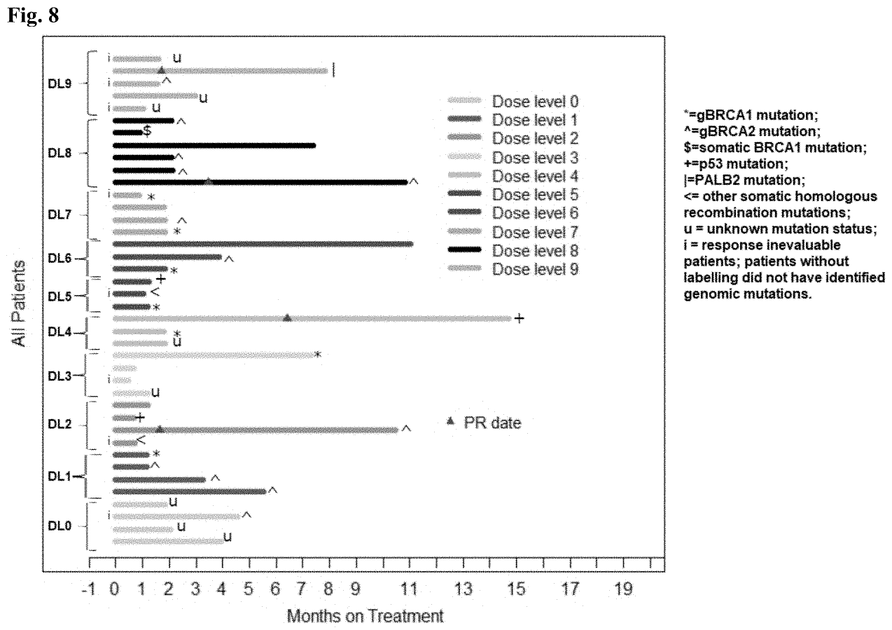

FIG. 7 shows % tumor shrinkage from baseline at each dose level in patients with genetic mutations in gBRCA1, gBRCA2, somatic BRCA, p53, PALB2 or other somatic homologous recombination mutations. Patients with unknown mutation status are labelled “u” in FIG. 7 and patients without labelling did not have identified genomic mutations. The duration on therapy at each dose level for evaluable patients is depicted in FIG. 8.

In the study described in Example 3, 18 patients were diagnosed with metastatic breast cancer. Of the 18 patients, 10 patients with metastatic breast cancer with BRCA12 germline and relevant somatic mutations, who did not receive prior PARP inhibitor treatments were enrolled in an ongoing study to assess predictability of biomarkers related to breast cancer. This study was conducted to evaluate predictive biomarkers of response to Compound I and to explore the relationship between germline HRD aberrations and outcomes of Compound I treatments.

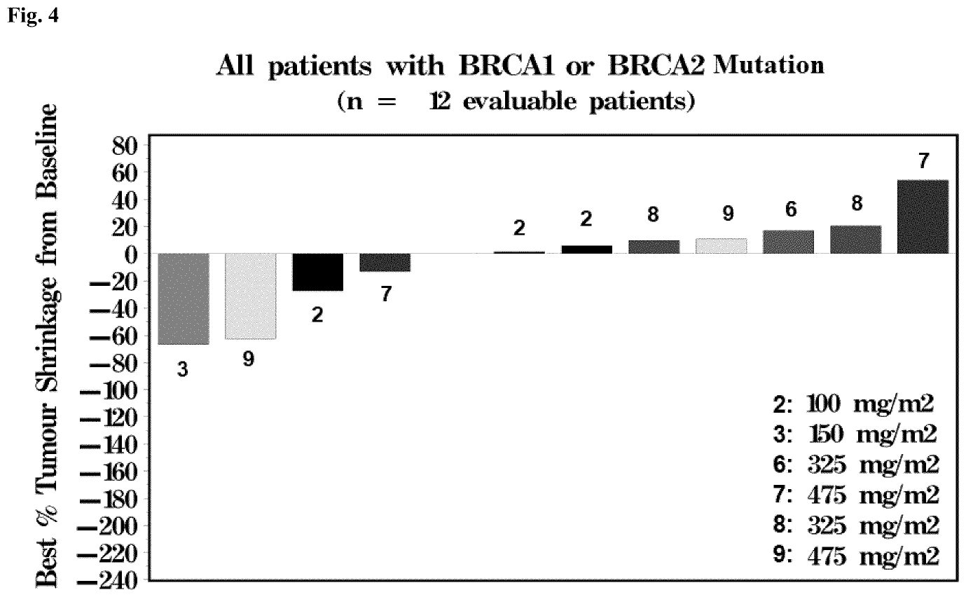

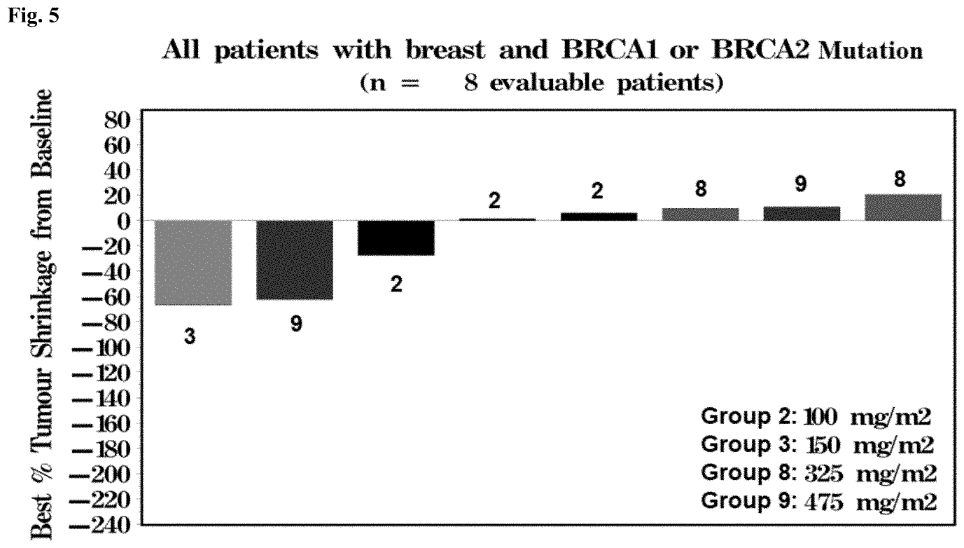

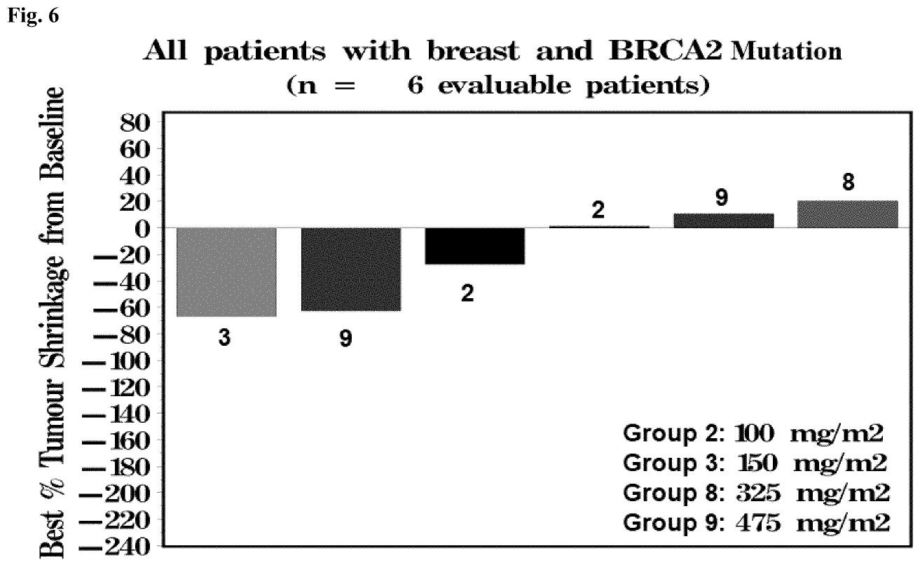

In the study described in Example 3, 12 patients harbored BRCA1 or BRCA2 mutation. The summary of these 12 patients tumor size during the treatment is shown in FIG. 4. From the same group of 12 patients harboring BRCA1 or BRCA2 mutation, 8 patients had breast cancer (FIG. 5). Of the 8 patients, 6 patient harbored BRCA2 mutation and had breast cancer (FIG. 6). In FIGS. 4-6, each bar represents an individual in a treatment Group as indicated by the Group number corresponding to the treatment Groups in Example 3.

One of the 10 patients enrolled in this study was from Group 10 (650 mg/m²), who harbored PALB2 mutation and BRCA2 mutation and showed partial response (PR) to Compound I treatment.

This study indicated that patients with BRCA2 mutation responded to Compound I treatment at a dose greater than or equal to 150 mg/m², where tumor shrinkage were observed.

### Example 5: Preparation of Liquid Formulation Comprising Compound I and Sucrose and Preparation of Lyophilized Form of Compound I with Sucrose for Injection 30 mg/mL (150 mg/Vial)

Composition:

| | Quantity per mL |
|---|---|
| Compound I | 30.0 mg |
| Sucrose | 20.0 mg |
| HCl | As needed, for pH adjustment |
| NaOH | As needed, for pH adjustment |
| Water for Injection (WFI) | q.s. |

Compounding: 37.5 kg of WFI was added to the compounding vessel at a temperature in the range of 15 to 30° C. Vigorously sparge WFI with nitrogen for no less than 30 minutes by placing the nitrogen sparging tubing at the bottom of the pressure vessel. Continue sparging in the vessel until dissolved oxygen content was ≤1 ppm. In a second pressure vessel was added 20.0 kg nitrogen sparged WFI and sucrose and mixed until dissolved while continuing nitrogen sparging, not less than 15 minutes. Nitrogen sparging continued as necessary until dissolved oxygen content was ≤1 ppm of the sucrose solution. To the sucrose solution, 813.8 g of 2 M HCl solution was slowly added and mixed for no less than 10 minutes after addition was complete. Add Compound I into the sucrose solution vessel and rinse container that contained Compound I with nitrogen sparged WFI. Mix solution until dissolved (no less than 15 min). Add 43.5 mL of 2 M HCl and mix for no less than 5 minutes. If solution is not visually dissolved, add another portion of 43.5 mL of 2M HCl and mix for no less than 5 minutes. Adjust pH, if necessary to 4.4-4.6 with 2M HCl or 1M NaOH solution prepared using nitrogen sparged WFI. Mix solution after each addition of 2M HCl or 1M NaOH. Adjust volume with nitrogen-sparged WFI as necessary. Pull a 10 mL sample to measure pH. If necessary, re-adjust pH to 4.4-4.6 using 2M HCl or 1M NaOH solution prepared using nitrogen sparged WFI. Filtration and filling directly proceeded this step. No material was stored overnight.

Sterilization through 0.22 μM membrane filters: A standard sterile filtration operation was designed to perform sterilization of the compounded bulk solution by membrane filtration through two 0.22 M hydrophilic polyvinylidene fluoride (PVDF) membranes contained in a polycarbonate housing. The compounded bulk passed through the two sterilizing membranes in series, as is typical in sterile filtration operations, to provide redundant sterilizing capability.

Aseptic filling of the sterile solution: The Compound I sterile solution was filled into 20-cc clean, de-pyrogenated glass vials, with periodic weight checks to assure that the target fill quantity (5.05 g/vial) was maintained, and the vials were semi-stoppered with sterile elastomeric closures to provide a sample of Formulation A. The filled vials are then transferred onto the shelves of the lyophilizer chamber for lyophilization to provide a sample of lyophilized Formulation B. Formulation B can be reconstituted for IV administration with solutions such as water, 5% dextrose in water, or 5% glucose in water.

The disclosures of all publications, patents, patent applications and published patent applications referred to herein by an identifying citation are hereby incorporated herein by reference in their entirety.

In the case of any conflict between a cited reference and this specification, the specification shall control. In describing embodiments of the present application, specific terminology is employed for the sake of clarity. However, the invention is not intended to be limited to the specific terminology so selected. Nothing in this specification should be considered as limiting the scope of the present invention. All examples presented are representative and non-limiting. The above-described embodiments may be modified or varied, without departing from the invention, as appreciated by those skilled in the art in light of the above teachings. It is therefore to be understood that, within the scope of the claims and their equivalents, the invention may be practiced otherwise than as specifically described.

1. A method for treating cancer in a subject, comprising administering to a subject in need thereof a therapeutically effective amount of Compound I, or a pharmaceutically acceptable salt or solvate thereof, 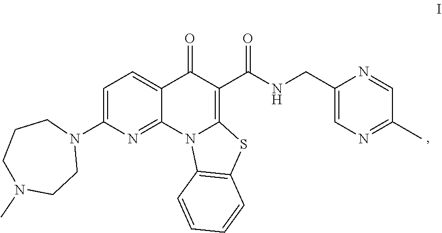
wherein the subject has a PALB2 mutation or a BRCA2 mutation.

2. The method of claim 1, wherein the subject has a PALB2 mutation.

3. The method of claim 1, wherein the subject has a BRCA2 mutation.

4. The method of claim 1, wherein the subject has a PALB2 mutation and a BRCA2 mutation.

5. (canceled)

6. The method of claim 1, wherein the cancer is a solid tumor.

7. The method of claim 1, wherein the cancer is a hematologic malignancy, colorectal cancer, breast cancer, lung cancer, liver cancer, ovarian cancer, cervical cancer, Ewing's sarcoma, pancreatic cancer, cancer of the lymph nodes, colon cancer, prostate cancer, brain cancer, bone cancer, cancer of the head and neck, skin cancer, kidney cancer, osteosarcoma, cancer of the heart, uterine cancer, gastrointestinal malignancies, or carcinomas of the larynx and oral cavity.

8. The method of claim 7, wherein the cancer is breast cancer, ovarian cancer, or pancreatic cancer.

9. The method of claim 7, wherein the hematologic malignancy is selected from leukemia, lymphoma, myeloma, and multiple myeloma.

10. The method of claim 1, wherein the cancer is a PALB2-mutated cancer.

11. The method of claim 1, wherein the cancer is a BRCA2-mutated cancer.

12. The method of claim 10, wherein the cancer is breast cancer, ovarian cancer, pancreatic cancer, or prostate cancer.

13. The method of claim 11, wherein the cancer is breast cancer, ovarian cancer, pancreatic cancer, or prostate cancer.

14. The method of claim 1, wherein the PALB2 mutation is a loss-of-function mutation of the PALB2 gene.

15. The method of claim 1, wherein the PALB2 mutation is a monoallelic loss-of-function mutation.

16. The method of claim 1, wherein the PALB2 mutation is a biallelic loss-of-function mutation.

17. The method of claim 1, wherein the BRCA2 mutation is a loss-of-function mutation of the BRCA2 gene.

18. The method of claim 1, wherein the subject is administered the Compound I or a pharmaceutically acceptable salt thereof at a dose ranging from about 50 mg to about 1,000 mg of the Compound I or a pharmaceutically acceptable salt and/or solvate thereof, per body surface area of the subject (m²).

19. (canceled)

20. The method of claim 1, wherein the dose ranges from about 150 mg to about 650 mg of the Compound I or a pharmaceutically acceptable salt and/or solvate thereof, per body surface area of the subject (m²).

21.-25. (canceled)

26. The method of claim 1, wherein the subject is administered the Compound I or a pharmaceutically acceptable salt and/or solvate thereof at a dose ranging from about 650 mg to about 800 mg of the Compound I or a pharmaceutically acceptable salt and/or solvate thereof, per body surface area of the subject (m²).

27.-30. (canceled)

31. claim 1, wherein the Compound I or a pharmaceutically acceptable salt thereof is administered intravenously.

32. claim 1, wherein the Compound I or a pharmaceutically acceptable salt thereof is administered in a 28-day cycle.

33. The method of claim 32, wherein a dose of the Compound I or a pharmaceutically acceptable salt thereof is:
a) administered on day 1, day 8, and day 15 of the 28-day cycle; or
b) administered on day 1 and day 8 of the 28-day cycle.

34. (canceled)

35. A pharmaceutical composition comprising Compound I, or a pharmaceutically acceptable salt and/or solvate thereof and a pharmaceutically acceptable carrier or excipient, 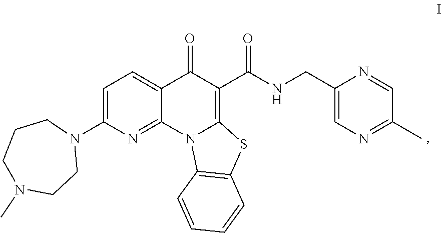
wherein, the composition provides a plasma Compound I AUC_{∞} ranging from about 2,000 ng*hr/mL to about 110,000 ng*hr/mL after a single dose administration of the composition to a human subject at a dose of about 50 mg to about 650 mg of the Compound I or a pharmaceutically acceptable salt and/or solvate thereof, per body surface area of the subject (m²).

36. The pharmaceutical composition of claim 35, wherein the dose of Compound I, or a pharmaceutically acceptable salt or solvate thereof ranges from about 150 mg² to about 650 mg of the Compound I or a pharmaceutically acceptable salt and/or solvate thereof, per body surface area of the subject (m²).

37.-43. (canceled)

44. The pharmaceutical composition of claim 35, wherein:
a) the plasma Compound I AUC_{∞} ranges from about 11,000 ng*hr/mL to about 52,000 ng*hr/mL after a single dose administration of the composition to a human subject at a dose of about 475 mg of the Compound I or a pharmaceutically acceptable salt and/or solvate thereof, per body surface area of the subject (m²); and/or
b) the plasma Compound I AUC_{∞} ranges from about 30,000 ng*hr/mL to about 40,000 ng*hr/mL after a single dose administration of the composition to a human subject at a dose of about 475 mg of the Compound I or a pharmaceutically acceptable salt and/or solvate thereof, per body surface area of the subject (m²); and/or
c) the plasma Compound I Cₘₐₓ ranges from about 900 ng/mL to about 2,600 ng/mL after a single dose administration of the composition to a human subject at a dose of about 475 mg of the Compound I or a pharmaceutically acceptable salt and/or solvate thereof, per body surface area of the subject (m²); and/or
d) the plasma Compound I Cₘₐₓ ranges from about 1,200 ng/mL to about 2,300 ng/mL after a single dose administration of the composition to a human subject at a dose of about 475 mg of the Compound I or a pharmaceutically acceptable salt and/or solvate thereof, per body surface area of the subject (m²); and/or
e) the plasma Compound I Tₘₐₓ ranges from about 1.0 hour to about 1.4 hour after a single dose administration of the composition to a human subject at a dose of about 475 mg of the Compound I or a pharmaceutically acceptable salt and/or solvate thereof, per body surface area of the subject (m²).

45.-52. (canceled)

53. The pharmaceutical composition of claim 35, wherein the composition comprises less than about 1% impurities.

54. The pharmaceutical composition of claim 35, wherein the composition comprises less than 0.1% of

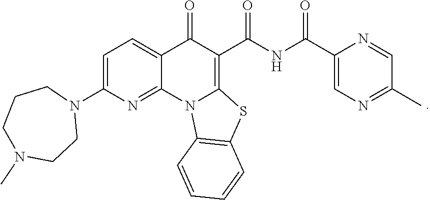

55. (canceled)

56. (canceled)

57. A method for treating or ameliorating cancer in a subject, comprising administering to the subject in need thereof a therapeutically effective amount of a pharmaceutical composition of claim 35.

58.-60. (canceled)

61. The method of claim 57, wherein the cancer is breast cancer, ovarian cancer, or pancreatic cancer.

62. The method of claim 57, wherein the hematologic malignancy is selected from leukemia, lymphoma, myeloma, and multiple myeloma.

63. The method of any one of claims 58-62 wherein the cancer is a PALB2-mutated cancer and/or a BRCA2-mutated cancer.

64.-81. (canceled)

82. The method of claim 57, wherein the composition is administered in a 28-day cycle.

83. The pharmaceutical composition of claim 82, wherein a dose of the Compound I or a pharmaceutically acceptable salt thereof is administered on day 1, day 8, and day 15 of the 28-day cycle, or on day 1 and day 8 of the 28-day cycle.

84.-88. (canceled)
